SEQUENCE LISTING (52 sequences)

SEQ ID NO: 1 - protein, 1880 aa, Monkeypox virus
MNFQKLSLAIYLTVTCSWCYETCMRKTALYHDIQLEHVEDNKDSVASLPYKYLQVVKQRERSRLLATFNWTDIAEGVRNEFIKICDINGTYLYNYTIAVSIIIDSTEELPTVTPITTYEPSIYNYTIDYSTVITTEELQVTPTYAPVTTPLPTSAVPYDQRSNNNVSTISIQVLSKILGVNETELTNYLIMHKNDTVDNNTMVDDETSDNNTLHGNIGFLEINNCYNVSVSDASFRITLVNDTSEEILLMLTGTSSSDTFISSTNITECLKTLINNVSINDVLITQNMNVTSNCDKCSMNLMASVIPAVNEFNNTLMKIGVKDDENNTVYKYYNCKLTTNSTCDELINLDEVINNITLTNIIRNSVSTTNSRKRRDLNGEFEFSTSKELDCLYESYGVNDDISHCFASPRRRRSDDKKEYMDMKLFDHAKKDLGIDSVIPRGTTHFQVGASGASGGVVGDSFPFQNVKSRASLLAEKIMPRVPITATEADLYATVNRQPKLPAGVKSTPFTEALVSTINQKLSNVREVTYASLNLPGSSGYVHRPSDSVIYSSIRRSRLPSDSDSDYEDIQTVVKEYNERYGRSVSRTQSSSSESDFEDIDTVVREYRQKYGNAMAKGRSSSPKPDPLYSTVKKTTKSLSTGVDIVTKQSDYSLLPDVNTGSSIVSPLTRKGATRRRPRRPTNDGLQSPNPPLRNPLPQHDDYYPPQVHRPPPLPPKPVQNPPQLPPRPVGQILPPPIDQPDKGFSKFVSPRRCRRASSGVICGMIQSKPNDDTYSLLQRPKIEPEYVEVGNGIPKNNVPVIGNKHSKKYTSTMSKISTKFDKSTAFGAAMLLTGQQAISQQTRSTTLSRKDQMSKEEKIFEAVTMSLSTIGSTLTSAGMTGGPKLMIAGMAITAITGIIDTIKDIYYMFSGQERPVDPVIKLFNKYAGLMSDNNKMGVRKCLTPGDDTLIYIAYRNDTSFKQNTDAMALYFLDVIDSEILYLNTSNLVLEYQLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATRDQFRNRWVLQNPFRQEGTYKQLFTFSKYDFNDTIIDPNGVVGHASFCTNRSSNQCFWSEPMILEDVSSCSSRTRKIYVKLGIFNAEGFNSFVLNCPTGSTPTYIKHKNADSNNVIIELPVGDYGTAKLYSATKPSRIAVFCTHNYDKRFKSDIIVLMFNKNSGIPFWSMYTGSVTSKNRMFATLARGMPFRSTYCDNRRRSGCYYAGIPFHEDSVETDIHYGPEIMLKETYDINSIDPRVITKSKTHFPAPLSVKFMVDNLGNGYDNPNSFWEDAKTKKRTYSAMTIKVLPCTVRNKNIDFGYNYGDIISNMVYLQSTSQDYGDGTKYTFKSVTRSDHECESSLDLTSKEVTVTCPAFSIPRNISTYEGLCFSVTTSKDHCATGIGWLKSSGYGKEDADKPRACFHHWNYYTLSLDYYCSYEDIWRSTWPDYDPCKSYIHIEYRDTWIESNVLQQPPYTFEFIHDNSNEYVDKEISNKLNDLYNEYKKIMEYSDGSLPASINRLAKALTSEGREIASVNIDGNLLDIAYQADKEKMADIQTRINDIIRDLFIHTLSDKDIKDIIESEEGKRCCIIDVKNNLVKKYYSIDNYLCDTLDDYIYTSVEYNKSYVLVNDTYMSYDYLESSGVVVLSCYEMTIISLDTKDAKDAIEDVIVASAVAEALNDMFKEFDKNVSAIIIKEEDNYLNSSPDIYHIIYIIGGTILLLLVIILILAIYIARNKYRTRKYEIMKYDNMSIKSEHHDSLETVSMEIIDNRY

SEQ ID NO: 2 - protein, 1840 aa, Monkeypox virus
MNFQKLSLAIYLTVTCSWCYETCMRKTALYHDIQLEHVEDNKDSVASLPYKYLQVVKQRERSRLLATFNWTDIAEGVRNEFIKICDINGTYLYNYTIAVSIIIDSTEELPTVTPITTYEPSIYNYTIDYSTVITTEELQVTPTYAPVTTPLPTSAVPYDQRSNNNVSTISIQVLSKILGVNETELTNYLIMHKNDTVDNNTMVDDETSDNNTLHGNIGFLEINNCYNVSVSDASFRITLVNDTSEEILLMLTGTSSSDTFISSTNITECLKTLINNVSINDVLITQNMNVTSNCDKCSMNLMASVIPAVNEFNNTLMKIGVKDDENNTVYKYYNCKLTTNSTCDELINLDEVINNITLTNIIRNSVSTTNSRKRRDLNGEFEFSTSKELDCLYESYGVNDDISHCFASPRRRRSDDKKEYMDMKLFDHAKKDLGIDSVIPRGTTHFQVGASGASGGVVGDSFPFQNVKSRASLLAEKIMPRVPITATEADLYATVNRQPKLPAGVKSTPFTEALVSTINQKLSNVREVTYASLNLPGSSGYVHRPSDSVIYSSIRRSRLPSDSDSDYEDIQTVVKEYNERYGRSVSRTQSSSSESDFEDIDTVVREYRQKYGNAMAKGRSSSPKPDPLYSTVKKTTKSLSTGVDIVTKQSDYSLLPDVNTGSSIVSPLTRKGATRRRPRRPTNDGLQSPNPPLRNPLPQHDDYYPPQVHRPPPLPPKPVQNPPQLPPRPVGQLLPPPIDQPDKGFSKFVSPRRCRRASSGVICGMIQSKPNDDTYSLLQRPKIEPEYVEVGNGIPKNNVPVIGNKHSKKYTSTMSKISTKFDKSTAFGAAMLLTGQQAISQQTRSTTLSRKDQMSKEEKIFEAVTMSLSTIGSTLTSAGMTGGPKLMIAGMAITAITGIIDTIKDIYYMFSGQERPVDPVIKLFNKYAGLMSDNNKMGVRKCLTPGDDTLIYIAYRNDTSFKQNTDAMALYFLDVIDSEILYLNTSNLVLEYQLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATRDQFRNRWVLQNPFRQEGTYKQLFTFSKYDFNDTIIDPNGVVGHASFCTNRSSNQCFWSEPMILEDVSSCSSRTRKIYVKLGIFNAEGFNSFVLNCPTGSTPTYIKHKNADSNNVIIELPVGDYGTAKLYSATKPSRIAVFCTHNYDKRFKSDIIVLMFNKNSGIPFWSMYTGSVTSKNRMFATLARGMPFRSTYCDNRRRSGCYYAGIPFHEDSVETDIHYGPEIMLKETYDINSIDPRVITKSKTHFPAPLSVKFMVDNLGNGYDNPNSFWEDAKTKKRTYSAMTIKVLPCTVRNKNIDFGYNYGDIISNMVYLQSTSQDYGDGTKYTFKSVTRSDHECESSLDLTSKEVTVTCPAFSIPRNISTYEGLCFSVTTSKDHCATGIGWLKSSGYGKEDADKPRACFHHWNYYTLSLDYYCSYEDIWRSTWPDYDPCKSYIHIEYRDTWIESNVLQQPPYTFEFIHDNSNEYVDKEISNKLNDLYNEYKKIMEYSDGSLPASINRLAKALTSEGREIASVNIDGNLLDIAYQADKEKMADIQTRINDIIRDLFIHTLSDKDIKDIIESEEGKRCCIIDVKNNLVKKYYSIDNYLCDTLDDYIYTSVEYNKSYVLVNDTYMSYDYLESSGVVVLSCYEMTIISLDTKDAKDAIEDVIVASAVAEALNDMFKEFDKNVSAIIIKEEDNYLNSSPDIYHIIYIIGGTILLLLVIILILAIYI

SEQ ID NO: 3 - protein, 1879 aa, Monkeypox virus
MNLQKLSLAIYLTVTCSWCYETCMRKTALYHDIQLEHVEDNKDSVASLPYKYLQVVKQRERSRLLATFNWTDIAEGVRNEFIKICDINGTYLYNYTIDVSIIIDSTEELPTVTPITTYEPSIYNYTIDYSTVITTEELQVTPTYAPVTTPLPTSAVPYDQRSNNNVSTISIQILSKILGVNETELTNYLIMHKNDTVDNNTMVDDETSDNNTLHGNIGFLEINNCYNVSVSDASFRITLVNDTSEEILLMLTGTSSSDTFISSTNITECLKTLINNVSINDVLITQNMNVTSNCDKCSMNLMASVIPAVNEFNNTLMKIGVKDDENNTVYNYYICKLTTNSTCDELINLDEVINNITLTNIIRNSVSTTNSRKRRDLNGEFEFSTSKELDCLYESYGVNDDISHCFASPRRRRSDDKKEYMDMKLFDHAKKDLGIDSVIPRGTTHFQVGASGASGGVVGDSFPFQNVKSRASLLAEKIMPRVPITATEADLYATVNRQPKLPAGVKSTPFTEALASTINQKLSNVREVTYASLNLPGSSGYVHRPSDSVIYSSIRRSRLPSDSDSDYEDIQTVVKEYNERYGRSVSRTQSSSSESDFEDIDTVVREYRQKYGNAMAKGRSSSPKPDPLYSTVKKTTKSLSTGVDIVTKQSDYSLLPDVNTGSSIVSPLTRKGATRRRPRRPTNDGLQSPNPPLRNPLPQHDDYSPPQVHRPPPLPPKPVQNPPQLPPRPVGQLPPPIDQPDKGFSKFVSPRRCRRASSGVICGMIQSKPNDDTYSLLQRPKIEPEYAEVGNGIPKNNVPVIGNKHSKKYTSTMSKISTKFDKSTAFGAAMLLTGQQAISQQTRSTTLSRKDQMSKEEKIFEAVTMSLSTIGSTLTSAGMTGGPKLMIAGMAITAITGIIDTIKDIYYMFSGQERPVDPVIKLFNKYAGLMSDNNKMGVRKCLTPGDDTLIYIAYRNDTSFKQNTDAMALYFLDVIDSEILYLNTSNLVLEYQLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATRDQFRNRWVLQNPFRQEGTYKQLFTFSKYDFNDTIIDPNGVVGHASFCTNRSSNQCFWSEPMILEDVSSCSSRTRKIYVKLGIFNAEGFNSFVLNCPTGSTPTYIKHKNADSNNVIIELPVGDYGTAKLYSATKPSRIAVFCTHNYDKRFKSDIIVLMFNKNSGIPFWSMYTGSVTSKNRMFTTLARGMPFRSTYCDNRRRSGCYYAGIPFHEDSVETDIHYGPEIMLKETYDINSIDPRVITKSKTHFPAPLSVKFMVDNLGNGYDNPNSFWEDAKTKKRTYSAMTIKVLPCTVRNKNIDFGYNYGDIISNMVYLQSTSQDYGDGTKYTFKSVTRSDHECESSLDLTSKEVTVTCPAFSIPRNISTYEGLCFSVTTSKDHCATGIGWLKSSGYGKEDADKPRACFHHWNYYTLSLDYYCSYEDIWRSTWPDYDPCKSYIHIEYRDTWIESNVLQQPPYTFEFIHDNSNEYVDKEISNKLNDLYNEYKKIMEYSDGSLPASINRLAKALTSEGREIASVNIDGNLLDIAYQADKEKMADIQTRINDIIRDLFIHTLSDKDIKDIIESEEGKRCCIIDVKNNRVKKYYSIDNYLCGTLDDYIYTSVEYNKSYVLVNDTYMSYDYLESSGVVVLSCYEMTIISLDTKDAKDAIEDVIVASAVAEALNDMFKEFDKNVSAIIIKEEDNYLNSSPDIYHIIYIIGGTILLLLVIILILAIYIARNKYRTRKYEIMKYDNMSIKSDHHDSLETVSMEIIDNRY

SEQ ID NO: 4 - protein, 1897 aa, Variola virus
MNLQRLSLAIYLTVTCSWCYETCMRKTALFHDNQLGHAEDNQDSVASLPYKYLQVVNKRERSRLLATFNWTSIAEGVKNDFIRICDINGTYLYNYTIAVSMIIDSMEELPTITTYEPSTYNYTFDNSTVSTTEELKVTPSPTTYATVTTPLPTSSVPYDQRSNNNVSTISIQILSKILGVNETELTNYLITHKNATVDNNTLYGNIGFLEINNCYNISVSNASFRITLVNNTSEEIVIMLTGTSSSDTFISSTNITECLKTLINNTSNISDVSITQNMNVTSNCDKCSMNLMTSVIPAVKEFNNTLKKIGVKDDKNNTVYNYYNCKLTTNSTCDELINLDEVINNITLTNIISSSVSTTNSRKRRDLNGEFEFSTSEELDCLYKSYGVSDDVSHCFSSPRRRRSDDKQEYTEMKLLDHAKKDLRIDSVIPRGTTHFQVGASGASGGVVGDSSPFQNVKSRASLLAEKIMPRVPTTATEEQLYATINRQTKLPAGVKSTPFTEALVSTINQKLSSVKEVTYASLNLPGSSGYVHRPSDSVIYSTIRRTRLPSDTDSDFEDIQTVVKEYNERYGRRVSRTQSSSSDFEDIDEVVAEYRQKYGGASRGRTSSSSSSDFEDIDEVVAEYRQKYGNAMTKGRGSSKPDPLYSTVKKTPKSIASGVDIVSKQTDYSLLPGVNTGSSIVTPLTRRGATRRPKRPSTPPREDLPPLPLNPPYRQLSRGGDHSLQQVPQRDYSPPHRPPPPLPPKPVPAIPPSRDSQPNNKGFSKFVSPRRCRRSTSGVVCGMIQSRPNDDTYSLLQLPKIEPEYAEVGNGLPKNNVPVIGNKHSKKYTSSMSKISTKFDKSMAFGTAMLLTGQQAINQQDRSTALIKKDQMSKDEKIFEAVTMTLSTIGSTLTTAGMIAPPLMIAGIGISLISGIIDTAKDIYYLFSGQEKPVDPVIKFFNTYAGLVSDSSKMGVRKCLTPGEDTLIYIAYKNDSSFKQNTEAMALYFLDVINSEILYLNTSNLVLEYHLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATWDQFRNRWVLQNPFRQEGTYKQLFTFSKYDFNDTIIDPNGVAGHASFCTNRSSNQCFWSEPMILEDVSSCSSRTRKIYVKLGIFNTEGFNSFVLNCPTGSTPTYIKDKNTDSNNVIIELPVGDYGTAKLYSVTKPSRIAVFCTHNYDKRFKSDIIVLIFNSISGIPFSSIYTGSVNGRNRLFTTLSKGMPYRSMYCDNRRPGCYYAGIPFNENSVESDLHYGPEIMLKETYDTNSIDPQVITKSKTHFPTPISVKFTVDNLGNGYNKPENFWKDAKSKKRTYSAMTIKILPCTVRNKNVDFGYNYGHIISNMVYAQSTSQDYGDGTNYTFKSVNRSDHECESILDLKAKEVTVMCPAFSIPRNISAYEGLCFSVTTSKDHCASNKEWLKSYGYGNTDATKQRVCFHHWNYVTTSLDYYCSYEDIWKSDWPDYDPCKSYIYIEYRDIWIESKVLQQPPYTFEFTHDDSNEYVNKEISNKLNDLYNEYKNIMEYSDGSLPASINRLAKSLTSEGREIASVNIDGNLLDIAYQADKEKMADIQNKINDITRDLFIHTLSNKDIKDIIESEEGKRCCIIDVKNNRVKKYYPIDNYLCGTLDDYIYTSVEYNKSYVLINDTYMSYDYLESSGVVVLSCYEMTIISLDTKDAKDAIEDEIVASAVAEALNDMFKEFDKNVSVIIIKEEDNYLNSSPNIYHIIYIIGGTILILLVIILILVIYIACNKYRTRKYKIMKDDTMSIKSEHHNSLETVSMEIMDNRY

SEQ ID NO: 5 - protein, 1869 aa, Camelpox virus
MNLQRLSLAIYLTATCSWCYETCMRKTALFHDNQLGHAEDNQDSVASLPYKYLQVVNKRERNRLLATFNWTSIAEGVRNDFIRICDINGTYLYNYTIAVSMIIDSTEELPIVTPITTYEPSTYNYTFDNSTVITTEEQLKVTPSPTTYATVTTPLPTSSVPYDQRSNNNVSTISIQILSKILGVNETELTNYLITHKNATVDNSTTNNNTLHGNIGFLEINNCYNISVSNASFRITLVNDTSEEIVLLLTGTSSSDTFISSTNITECLKTLINNTSNISDVSITQNMNVTSNCDKCSMNLMTSVIPTVKEFNNTLKKIGVKDDKNNTVYNYYNCKLTTNSTCDELINLDEVINNITLTNIISSSVSTTNSRKRRDLNGEFEFSTSKELDCIYESYGVSDDVSHCFSSPRRRRSDDKQEYTEMKLLDHAKKDLRIDSVIPRGTTHFQVGASGSSGGVVGDSSPFQNVKSRASLLVEKIMPRVPTTATEEQLYATINIQTKLPAGVKSTPFTEALVSTINQKLSSVKEVTYASLNLPGSSGYVHRQSDSVIYSTIRRTRLPSDSNSDFEDIQTVVKEYNERYGRRVSRTQSSSSDFEDIDEVVAEYRQKYGNAMTKGRGSPKPDPLYSTVKKTPKSIVSGVDIVSKQTDYSLLPGVNTGSSIVTPLTRRGATRRPKRPSTPPREDLPPLPPNPPRRQLPRGGDHSPPQVPQRDYSPPLPPKPVPAIPPRDGQPDNKGFSKFVSPRRCRRSTSGVVCGMIQSRPNDDTYSLLQRPKIEPEYAEVGNGLPKNNVPVIGNKHSKKYTSSMSKISTKFDKSMAFGTAMLLTGQQAINQQVRSTELIKKDQMSKDEKIFEAVTMTLSTIGSTLTTAGMIAPPLMIAGIGISLISGIIDTAKDIYYLFLGQEKPVDPVIKFFNTYAGLVSDSSKMGVRKCLTPGEDTLIYIAYKNDSSFKQNTEAMALYFLDVIDSEILYLNTSNLVLEYQLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATWDQIRNRWVLQNPFRQEGTYKQLFTFSKYDFNDTIIDPNGVAGHASFCTNRSSNQCFWSEPMILEDVSSCSSRTRKIYVKLGIFNAEGFNSFVLNCPTGSTPTYIKDKNADSNNVIIELPVGDYGTAKLYSATKPSRIAVFCTHNYDKRFKSDIIVLIFNSISGIPFSSIYTGSVNGRNRLFNTLSRGMPYRSIYCDNRRPGCYYAGIPFNENSVESDLHYGPEIMLKETYDANSIDPLVITKSKTYFPTPISVKFTVDNLGNGYNKPENFWKDAKSKKRTYSAITIKILPCTVRNKNVDFGYNYGHIISNMVYVQSTSQDYGDGTNYTFKSVNRSDHECESILDLKAKEVTVMCPAFSIPRNISAYEGLCFSVTTSKDHCASNKEWLKSYGYGKADATKQRVCFHHWNYITTSLDYYCSYEDIWKSDWPDYDPCKSYIHIEYRDIWIESKVLQQPPYTFEFTHDDSNEYVNKEISNKLNDLYNEYKNIMEYSDGSLPASINRLAKSLTSEGREIASVNIDGNLLDIAYQADKEKMADIQNKINDITRDLFIHTLSNKDIKDIIESEEGKRCCIIDVKNNRVEKYYPIDNYLCGTLDDYIYTSVEYNKSYVLVNDTYMSYDYLESSGVVVLSCYEMTIISLDTKDAKDAIEDEIVASAVAEALNDMFKEFDKNVSAIIIKEEDNYLNSSPNIYHIIYIIGGTILILLVIILILAIYIARNKYRTRKYKIMKDDTMSIKSEHHNSLETVSIEIMDNRY

SEQ ID NO: 6 - protein, 1919 aa, Cowpox virus
MNLQRLSLAIYLTATCSWCYETCVRKSALYHDNQLGHAEDNQDSVASLPYKYLQVVNQRERSRLLATFNWTSIAEGVRNEFIKICDINGTYLYNYTIAVSMTIDSTEELPTVTPYTTYEPSTYNYTIDNGTVVTTEELKVTPSPTPYATVTTPLPTSSVPYDQRSNNNVSTISIQILSKILGVNETELTNYLITHKNVTVDNNTTNNNITVNDETSDNNTLHGNIGFLEINNCYNISVSNASFRITLVNDTSEEIVLMLTGTSSSDTFISSTNITECLKTLINNTSNISDVSITQNMNVTSNCDKCSMNLMTSVIPVVNEFNNTLEKIGVKDDKNNTVHNYYNCKLTTNSACDELINLDEVINNITLTNIISSSVSTTNSRKRRDLNGEFEFSTSKELDCLYESYGVSDDVSHCFSSPRRRRSDDKQEYTEMKLLDHAKKDLGIDSVIPRGTTHFQVGASGASGGVVGDSNPFQNVKSRASILAEKIMPRVPTTATEEQLYATVNRQAKLPAGVKSTPFTEALVSTINQKLSSVKEVTYASLNLPGSSGYIHRPSDSVIYSTIRRTRLPSDSDSDFEDIQTVVKEYNERYGRRVSRTQSSSSSDFEDIDEVVAEYKQKYGGAAASRGRTSSSSSSDFEDIDEVVREYNQKYGTAMTKGRGSPKPDPLYSTVKKTPKSIASGVDIVTKQTDYSLLPGVNTGSSIVTPLTRRGATRRPKRPSTPPREDLPPLPPNPPRRQLPRGGDHSPPQVPQRDYSPPLPPRGPPPLPPKPVPAIPPRDGQPDNKGFSKFVSPRRCRRSTSGVVCGMIQSRPNDDYSLLQRPKIEPEYAEVGNGLPKNNVPVIGNKHSKKYTSAMSKISTRFDKSMAFGTAMLLTGQQAINQQARSTALIRKDQMSKDEKIFEAVTMTLSTIGSTLTTAGMIAPPLMIAGIGITAITGIIDTVKDIYYLFSGHEKPVDPVVKLFNTYAGLVSDSNKMGVRKCLTPGEDTIIYMAYRNDTSFKQNTEAMALYFLDVIDSEILYLNTSNLVLEYQLRVACPIGTLRSVDVDITAYTILYDTADNIKKYKFVRLATLLSKHPVIRLTCGLAATLVIKPYEVPISDMQLLKMATPGEPESTKSIPSDVCDKYPLKKFYLLAGGCPYDTSQTFIVHTTCSILLRTATWDQFRNRWVLQNPFRQEGAYKQLFTFSKYDFNDTIIDPNGVAGHASFCTNRSSNQCFWSEPMILEDVSSCSSRTRKIYVKLGIFNAEGFNSFVLNCPTGSTPTYIKDKNADSNNVIIELPVGDYGTAKLYSATKQSRIAVFCTHNYDKRFKSDIIVLIFNSISGVPFSSIYTGSVNGRNRLFTTLSRGMPYRSMYCDNRRPGCYYSGIPFNENSVESDLHYGPEIMLKETYDANSIDPRVITKSKTHFPTPISVKFMVDNLGNGYNKPENFWKDAKNKKRTYSAMTIKILPCTVRNKNVNFGYNYGHIISNMVYAQSTSQDYGDGTNYTFKSVNRSDHECKSILDLKAKEVTVMCPAFSIPRNISAYEGLCFSVTTSKDHCATDNDWLKSHGYGKADAIKQRACFHHWNYATTSLDYYCSSENLFKSDWPDYDPCKSYIHIEYRDIWIESKVLQQPPYTFEFTHDDSNEYVNKEISNKLNDLYNEYKNIMEYSDGSLPASINRLAKALTSEGREIASVNIDGNLLDIAYQADKEKMADIQNKINDITRDLFIHTLSDKDIKDIIESEEGKRCCIIDVKNNRVEKYYPIDNYLCGTLDDYIYTSVEYNKSYVLVNDTYMSYDYLESSGVVVLSCYEMTIISLDTKDAKDAIEDEIVASAVAEALNDMFKEFDKNVSAIIIKEEDNYLNSSPNIYHIIYIIGGTILILLVIILILAIYIARNKYRTRKYKIMKDDTMSIKSEHHNSLETVSMEIMDNRY

SEQ ID NO: 7 - protein, 1924 aa, Ectromelia virus
MNLQKLSLAIYLTATCSWCYETCMRKTALFHDNNLEHVEENHDSGVASLPYKYLQVVHQRERSRLLATFNWTSIAEEVKNEFIKICDINGTYIYNYTITVSMIIDSTEELPTVTPFTTYEPSTYNYTFDNSTVSTTEELKVTPSPTPYATVTTPLPTSSVPYDQRSNNNVSIISIQILSKILGVNETELTNYLITHKNAMVDNNTINNNITVNSTTVYDDTSDNNTLHGNIGFLEINNCYNVSVSDASFRITLVNDSSEEIVLMLTGTSSSDTFISSTNITECLKTLINNTSNISDVSITQNMNVTSNCDKCSMNLMTSVIPVVNEFNNTLEKIGVKDDKNNTVHNYYNCKLTTNSTCDELINLDEVINNITLTNIISSSASTINNRKRRDLNGEFEFSTSKELDCLYEAYGVNEDISHCFASPRHRRSDDKQEFIEMKLLDHAKKDLSIDSVIPRGTTHFQVGASGASGGVVGDSNPFQNVKSRASLLAEKIMPRVPTTATEDQLYATINRQAKLPAGVKSTPFTEAVVSTINQKLSSVKEVTYASLNLPKSSGYIHRPSESVIYRTIRRTRLPSDSDSDFEDIQTVVKEYNERYGRRVSRTQSSSSSDFENIDEVVAEYKQKYGGAASRVRTSSSSSSDFENIDEVVAEYNQKYGNAMTKGRGSLKPDPLYSTVKKTPKSIASGVDIVSKQTDYSLLPGVNTGSSIVTPLTRRGATRRPKRPSTPPREDLPPLPPNPPHRQLPRGGDHSLPQVPQRDYSPPRRPPPPLPPKPVPAIPPRDGQPDNKGFSKFVSPRRCRRSTSGVLCGMIQSRPNDDVYSLLQRPKIEPEYAEIGNGLPKNNVPVIGKKHSKKYKSSMTKISTKFDKSMAFGTAMLLTGQQAINQQVRSTALIRKDQMSKDEKIFEAVTMTLSTIGSTLTTAGMISPPLMIAGMGISLISGIIDTVKDIYYLFSGHEKPVDPVIKFFNTYAGLVSDSSKMGVRKCLTPGEDTLIYIAYRNDTSFKQNTEAMALYFLDVIDSEILYLNTSNLVLEYQLKVACPIGTLRSVDVDITAYTILYDTADNIKKYKFVRLATLLSKHPVIRLTCGLAATLVIKPYEVPISDMQLLKMATHGEPESTKSIPSDVCDKYPLKKFYLLAGGCPYDTSQTFIVHTTCSILLRTATWDQFRNRWVLQNPFRQEGTYKQLFTFSKYDFNDTIIDPNGVAGHASFCTNRSSNQCFWSEPMILEDVSSCSSRTRKIYVKLGIFNAEGFNSFVLNCPTGSTPTYIKDKNADSNNVIIELPVGDYGTAKLYSATKQSRIAVFCTHNYDKRFKSDIIVLIFNSISDIRFSSIYTGDVNGRNRLFITLSRGMPYRSMYCDNRRPGCYYAGIPFNENSVESDLHYGPEIMLKETYDANSIDPRVITKSKTHFPTPISVKFMVANLGNGYNKPENFWNDAKSKKRTYSAMTIKILPCTVRNKNVNFGYNYGHIISNMVYAQSTSYDYGDGTNYTFKSVNRSDHECESILDLKAKEVTVTCPAFSIPRNISAYEGLCFSVTTSKDHCATDNNWLKSHGYGKADAIKQRACFHHWNYVTTSLDYYCSAENVFKSYWPDYDPCKSYIHIEYRDIWIESNVLQQPPYTFEFTHDNSNEYVDKEISNKLNDLYNEYKKIMEYSDGSLPASINRLAKALTSEGREIASVNIDGNLLDIAYQADKEKMAEIQNKINDITRDLFIHTLSDKDIKDIIESEEGKRCCIIDVKNNRVEKYYPIDNYLCGTLDDYIYTSVETNKSYVLVNDTYMSYDYLESSGVVVLSCYEMTIISLDTKDAKDAIEDEIVASAVAEALNDMFKEFDKNVSVIIIKEEDNYLNSSSDIYNILYIICGAILLLLVIILILAIYIARNKYRTRKYKMMKDYDNRSIKSEHHNSLETVSMEIMDNRY

SEQ ID NO: 8 - protein, 2133 aa, Molluscum contagiosum virus
MSIEAMSSADGLSGPGFTISIIYALEIAPAGKVNFYRHTWSGRIMSPRGDLTARCAVPLVALLTILFAALFAAGSGTEMACYRKIGLYSLTGEMHRAQETRVVSVLDVDSRVLAEMETLQEGFHWDALRSNASARFRSACENTSTPQVSYVVRQGLTLLTRANCSDDGNSTDILSGLNGTLLVTVCNMTLELQQQDCPATMQGLELSVHEGSVTLTLVSGLVSSVNPFVPSENATRCLMAVPTKITYNGSCENIPLSTPTSMPEGTPPGASADTLKNTESTPGNASADERPLASTPAPLSTPASFFATTLEISSGTRAVTVAWNLASTPTLTPASMSFEEQHVVWTNLTVEDNCTIMVGIQVKSSAQSACERADMETVLAVVGVLTEYTEALHRLNLSSDNETKDYYECMMLGSPSCFLQGLRQVALHNVMQNLQAKALTRARRTRSLHDDNAHCLHRHFGMGPGVGDHCENIEEPLARVRRSDPPRPTPPRVRRPRPGGVDTPQRPLPKPRPGGGSTPPIPPTKPKSLVEKVSESLGMKPVIGPGVQEVQLGAHGSDGSVVGSDGLTTSLREQLRKAVEQRAPTLPTDMNPDDLEKARIRWREGGGHLQGLCSEAVRYKEESIRSAAGKAHDWSKRKHRWGQRRGFHGTRGGGHGDEDSDLEDVEQVRQQLKEMGIGPLGRQRSSGSSSDLEDVEEVRQQLKEMGIGPLGKRKSSSSSNSDFEHLQDTQQQLVKKGLFRGGRQSGPTRFDESVWKKLDQLGGPAHAPVFSPQVHGSPPGDVVAPLETRMSGDPGHLDVHSSGRHTPTFPNLDTGTRVSVAETTYAVLGGGGGPRPPRRLRERQREGGDGVGDYAELDWGEQRHAGRRRGHGRGVQDGSGPLPPIPGEKSPTGRQLGDRPLPPTPTSRHEDSPGTRLGRKICKRSLDSLLCGMLGTRPDSDSVPHDPVYESVQGPYSLLGEHNRPPATNPNYREPVYSTLGMPTGPEAPSKTGGGPSAPPTELMPADTSSPRMKQLAREENRLEKFLSAVSLSTYLSSAQTQLREVMMVQPHMPHGMAVATMISSIVSTAGGTLALAGASNPVTAAAGLALQGVGMLIDIGTSLYYLIKGQSRPPPVDPVTEKFSTYARHMAQASAGARLCLMPDSDLRLTLAYRHSHFESAAGEKGALAFADTPMTLVYYLRSQYIVYNTRVTVTCPIGTLRLLEGDISAYAVLESVGEDGASHYALPGIMELLSNHPNASFTCGSEVGARFVPFDRELGDMQLLRVAAPGEPKETESIPSNVCDLFPLKRFYLLTDGCPHDRSQTAITYVTCGTLLRMASWEPVRNRWVLLNPFFRSADEFIQLFTFSQYDFSGSTIRLHELDAPEAFCSQRVSSTCYWPEPMMLEDVTACETRIRKIHVELATVGGSGYTSFVLTCPPGSTPFHVSNVSVVAIPQNTRRTAVRYAATTQTSILVSCVHNTNPAYKSDIVVLTFSTQGLRSRFSDMEKWTQRKMLFDSSAWAMPLRSRTCKRPEENTLCHLAYSVSQQRAEYHLQVQTLPEQRLHEYYHGELDALTLQLTRTHFASDLHLRVDVSFLASAYRTPENLWKHAAKQMRTFSAITITLYPCASMVGMFDIKYEQVLARLLYLGTKDHGNGQNLTFHALNSLNDSRHARHGHELHADYSCRAHLDIPEKTVSLHCPPLSLPKAPFNDTGVDGICVVVTSSRDHCAVQSEEGVREGYTSYEAETPFLSCGAYVQEYAVRENFCYYERTLRIPLAHDYDPCSTAMVLGYAHVWLETRLVSPPYVQEFGYDPSRNEYANRSLFEQLQDLQRQYEELLKHSSNPAVRMANQLAATLTEEARAIFRINADSQLMQTAFEADEERLRELEARIENTMQDIFLNTMSYQELDALRRSAFSTRCCVLDGTSVRKYFPLEHYLCGNYGDYIVTAGEYRFLLINHTLVDEAYYNATEQPWLTCYEITLVPVSTEEQRARVEEALFLDALESVLQDLFNRYDENLTVILHNPGLGKHDEEEDGGTSNDNGPGGTGLSVPLGVSLATAMLVLLAFLLARRGYAAKCMGKYSPLKAQQKQVRDFWKQVCERYSAREEAGISGELARSASFYERRSSDGGHFARRSSSALLLRQEETSA

SEQ ID NO: 9 - DNA, 5642 bp, Artificial Sequence
  Codon optimized MPXV197 nucleotide sequence
atgaacttccagaagctgtctctggccatctacctgactgtgacctgcagttggtgttatgagacatgcatgcggaagactgccctgtaccacgacatccagctggagcatgtcgaagataacaaggacagtgtggcttcactgccttacaaatatctgcaggtggtcaagcagagagagcgcagccgactgctggctactttcaactggaccgacatcgcagagggggtgagaaacgagttcatcaagatctgtgatatcaacggtacttacctgtacaactacaccatcgctgtgtccatcattatcgacagcaccgaggaactgcctacagtcactccaattaccacatacgagccaagcatctataactacaccatcgattactctacagtgatcactaccgaggaactgcaggtcacccccacatacgctcctgtgacaactcccctgcctaccagtgcagtgccctatgaccagcggtcaaacaataacgtctccacaatcagcattcaggtgctgtctaagattctgggcgtcaacgagaccgaactgacaaattacctgatcatgcacaaaaacgatactgtcgacaataacaccatggtggacgatgagacatccgacaataacactctgcatgggaacatcggtttcctggaaattaataactgctacaacgtctcagtgtccgacgcctccttcaggattaccctggtgaatgatacatccgaggaaatcctgctgatgctgactggcacctccagctctgatacttttatcagttcaactaacatcaccgagtgtctgaagaccctgatcaataacgtctctattaatgacgtgctgatcacacagaatatgaacgtgactagtaactgcgataagtgttcaatgaatctgatggcttccgtcattccagcagtgaacgagttcaataacacactgatgaagatcggagtgaaagacgatgaaaacaacactgtctacaagtactacaactgcaaactgaccacaaatagcacctgtgacgagctgatcaatctggatgaagtgatcaataacatcacactgactaacatcatccgtaacagcgtctctactaccaactctaggaagaggcgggacctgaatggcgagttcgagttctccacctcaaaagagctggattgcctgtatgaatcctacggagtgaacgacgatatctctcactgtttcgcaagtcccagacgccgacgtagcgacgataagaaagagtacatggacatgaagctgtttgatcacgccaagaaagacctggggatcgattccgtgattcctcgcggtacaactcatttccaggtcggagcatctggagcaagtggaggagtggtcggggacagcttcccatttcagaacgtgaagtcacgcgcctccctgctggctgagaaaattatgccccgagtgcctatcaccgctacagaagcagatctgtacgccaccgtgaatcgccagccaaaactgcccgctggagtcaagtctacaccctttactgaggcactggtgtcaaccatcaatcagaagctgtccaacgtccgagaagtgacatacgcatccctgaacctgcctgggtccagcggttacgtgcatcgcccatccgacagcgtcatctattctagtattaggcggagccgactgccttctgacagtgattcagactatgaggatatccagaccgtggtcaaggagtataacgaacgttacggcaggtccgtgagcaggactcagtcatccagctctgagagcgacttcgaagatatcgacaccgtggtccgggagtatagacagaaatacgggaatgctatggcaaagggtcggagttcatccccaaaacccgaccctctgtactctaccgtgaagaaaaccacaaagtctctgagtaccggggtcgatattgtgacaaaacagagcgactattctctgctgccagatgtgaacaccggcagctctatcgtcagccccctgactaggaagggagcaaccagacgccgacctcgtaggccaacaaatgacggtctgcagtctcctaatccacctctgcgaaacccactgccacagcacgacgattactatccaccccaggtccatagacctccacccctgcctccaaagcctgtgcagaacccacctcagctgccaccacgaccagtcggacagattctgcctccacccatcgatcagcccgacaagggattctccaaatttgtgagccctcggagatgccgccgagctagttcaggcgtcatctgtggaatgattcagtccaagccaaacgacgatacctacagcctgctgcagaggccaaaaatcgagcccgaatatgtcgaagtgggcaacggaattccaaagaataacgtccccgtgatcggcaacaagcacagtaagaaatacactagtaccatgtcaaagatttccactaagttcgacaaatccaccgcctttggggccgctatgctgctgactggtcagcaggctatcagccagcagacccggtctactaccctgagtagaaaggatcagatgtctaaggaggaaaaaatttttgaggcagtgacaatgtcactgtccactatcggctcaacactgacttccgccggaatgaccgggggtccaaagctgatgatcgccggcatggctatcaccgcaattacaggaattatcgacacaatcaaggatatctactatatgttctctggacaggagcgaccagtcgaccctgtgatcaagctgtttaacaaatacgccggtctgatgtccgataataacaagatgggggtgcgcaaatgcctgacccccggtgacgatacactgatctatattgcttaccgaaacgacacaagcttcaagcagaatactgatgccatggctctgtactttctggatgtgatcgactctgagattctgtacctgaataccagtaacctggtgctggaatatcagctgaaggtcgcctgtcctatcggcacactgcgctctgtcgatgtggacattactgcctataccatcctgtacgatacagctgacaacattaagaaatacaagttcatccgtatggctactctgctgtctaaacacccagtgatcaggctgacctgcggactggcagcaacactggtcattaagccttatgaggtgccaatctccgacatgcagctgctgaagatggccaccccaggcgagcccgaaagcacaaaatctatcccaagtgatgtgtgtgacaggtaccccctgaagaaattctatctgctggctggcggatgcccctacgacacaagccagacttttattgtgcacacaacttgttctatcctgctgcgcaccgcaacacgagatcagttccgtaacagatgggtgctgcagaatccttttagacaggagggcacttacaagcagctgttcaccttttctaagtacgatttcaacgacactatcatcgaccccaatggcgtggtcggacatgccagcttctgcaccaatcgctccagcaaccagtgtttttggtctgagcctatgatcctggaagatgtgtctagttgctcatcccggaccagaaagatctacgtcaaactgggcatcttcaatgccgagggattcaacagttttgtgctgaattgtcctaccggatcaactccaacctatatcaagcacaaaaacgctgactctaataacgtcattatcgaactgccagtgggggattacggtactgccaaactgtattcagctaccaagccctcccggatcgctgtgttctgcacccataattacgacaagaggttcaagtccgatattatcgtgctgatgttcaataagaacagcggcatccctttttggtctatgtacacaggatcagtgacttccaaaaaccgcatgttcgccacactggctcgtggcatgcccttcaggtccacttattgcgacaatcgtaggcggagcggctgttactatgccggaatcccctttcacgaggactccgtggaaaccgatattcattacggacctgagatcatgctgaaggaaacatatgacatcaactctattgatcccagagtgatcaccaagagtaaaacacacttccctgctccactgtcagtcaagtttatggtggacaatctggggaacggttacgataatccaaacagtttctgggaggatgcaaagactaagaaacgtacctattccgccatgacaattaaggtcctgccctgcactgtgaggaacaagaacatcgactttgggtataactacggcgacattatctccaatatggtgtacctgcagagcacctctcaggattacggcgacggaaccaagtatacattcaaaagcgtgactcggtctgaccacgagtgcgaaagctctctggatctgaccagcaaggaggtcacagtgacttgtcccgcattcagtatccctagaaacatttcaacctacgaaggcctgtgctttagtgtgaccacatcaaaggaccattgtgctacagggatcggttggctgaaaagttcaggctatggaaaggaggatgcagacaaacccagggcctgctttcaccattggaactactatacactgtccctggactactattgtagctacgaagatatctggcgtagtacttggcccgattatgacccttgtaagtcatatatccacattgagtacagggatacatggatcgaatccaatgtgctgcagcagcctccatacactttcgagtttattcatgacaattccaacgagtatgtggataaggaaatcagcaacaagctgaacgacctgtacaacgagtacaagaaaattatggaatacagcgatggctctctgcctgctagcatcaatcgactggcaaaggcactgactagcgagggaagagaaattgcatctgtgaatatcgacggtaacctgctggatatcgcatatcaggccgacaaggagaaaatggccgatatccagacccgcattaacgacattatccgagatctgttcattcacacactgagtgataaggacatcaaagacattatcgaatccgaggaaggcaagagatgctgtattatcgatgtcaagaacaacctggtgaagaaatactactccattgacaattacctgtgcgataccctggacgattatatctacacatctgtggagtataacaagagttacgtcctggtgaatgacacctacatgagctatgattacctggagtccagcggagtggtcgtgctgtcatgttatgaaatgacaattatctccctggatactaaggacgctaaagatgcaatcgaggacgtcattgtggctagcgcagtggccgaggctctgaacgacatgttcaaggaattcgataagaacgtgtctgctatcatcattaaggaggaagacaattacctgaactctagtcccgatatctatcatatcatctacatcattgggggtaccattctgctgctgctggtcatcattctgatcctggcaatctatattgcccgaaacaagtatcgcacacgaaagtacgagatcatgaagtacgacaacatgagtatcaagtcagaacaccatgatagcctggagaccgtgtctatggaaatcattgacaacagatataa

SEQ ID NO: 10 - DNA, 5694 bp, Artificial Sequence
  Codon optimized nucleic acid sequence of Variola virus B22R
atgaatctgcagcgactgagcctggcaatctatctgaccgtgacttgttcctggtgttacgagacttgtatgcgaaagaccgccctgttccacgacaatcagctgggccatgcagaggataaccaggactctgtggccagtctgccctacaagtatctgcaggtggtcaacaaacgggagagatcaaggctgctggctaccttcaattggacaagcatcgcagaaggggtgaagaacgactttattaggatctgcgatatcaatggaacctacctgtataactacacaattgccgtgagcatgatcattgacagcatggaggaactgccaaccatcaccacatacgagccctccacttacaactacacctttgataactcaactgtcagcactaccgaggaactgaaggtgaccccatctcccacaacttatgccacagtcaccacacctctgccaactagctccgtgccctacgaccagcggagcaacaataacgtgtccaccatttctatccagattctgagcaagatcctgggcgtgaatgagaccgaactgacaaactacctgattacccacaaaaatgccacagtggataataacactctgtatgggaacatcggattcctggagattaataactgctacaacatcagcgtgtccaatgcttcctttcggattaccctggtcaataacacatctgaggaaatcgtgattatgctgactgggacctctagttcagacaccttcatcagctccactaatattaccgaatgtctgaagactctgatcaataacaccagtaacatctcagacgtgagcattactcagaatatgaacgtgaccagcaattgcgataagtgttccatgaacctgatgacttctgtcatccccgccgtgaaggagtttaataacaccctgaagaaaattggcgtcaaggacgataagaacaacacagtgtacaactactacaactgcaagctgactaccaattccacttgtgacgagctgatcaacctggatgaagtgatcaataacattacactgactaacatcatttctagttcagtgtcaacaactaatagccggaagcggagagacctgaacggggagttcgaattttctaccagtgaggaactggattgcctgtataaatcctacggagtctctgacgatgtgagtcactgtttcagctcccctaggcgccgacggtctgacgataagcaggagtacaccgaaatgaaactgctggaccacgccaagaaagacctgaggatcgatagcgtcattcctcgcggaaccacacatttccaagtgggagcatccggagcatctggaggggtggtcggcgattctagtccatttcagaatgtgaagagtagggcttcactgctggcagagaaaatcatgccccgcgtgcctactaccgcaaccgaggaacagctgtacgctacaattaacagacagactaagctgcctgctggggtcaaaagcacacccttcaccgaggcactggtgtccacaatcaatcagaagctgtcaagcgtcaaagaagtgacttacgcaagcctgaacctgcctggcagcagcggctatgtccatcgcccatcagacagcgtgatctactccacaattagaaggactcggctgccatcagacaccgatagcgacttcgaggatatccagacagtggtcaaggagtataacgaacgatacggccgccgagtgtcccggacccagagttcaagctccgactttgaggatattgacgaagtggtcgcagagtataggcagaagtacggaggagccagccgcggacgaacctctagttcaagctcctctgacttcgaagatatcgacgaggtggtcgctgaatatcgacagaaatacggcaacgcaatgacaaaggggaggggaagttcaaaacccgatcctctgtatagcaccgtgaagaaaacacctaagagcatcgcatccggagtcgacattgtgagtaaacagacagattactcactgctgcccggagtcaatactggcagctccatcgtgacccctctgacacggagaggagcaactaggcgcccaaagcgaccatccaccccacctagagaggacctgccaccactgccactgaaccctccatatcgacagctgagccgagggggagaccactccctgcagcaggtgccacagagagattacagcccacctcataggccaccacctccactgccacctaagccagtgccagctatcccaccctctcgcgacagtcagccaaataacaaggggttctccaaattcgtgagcccccgacggtgcagaaggtccacctctggggtggtctgtggaatgatccagtcacggcccaacgacgatacatatagcctgctgcagctgccaaagattgagcccgaatacgccgaagtgggcaatgggctgccaaagaataacgtccccgtgatcggaaacaaacacagcaagaaatacacatctagtatgtcaaagatcagcactaagttcgacaaatccatggcctttggaaccgctatgctgctgacaggccagcaggccatcaaccagcaggatagaagtaccgctctgattaagaaagaccagatgtcaaaggatgagaaaatcttcgaagccgtgaccatgacactgagtactattgggtcaaccctgacaactgcaggaatgatcgcccctccactgatgattgcaggaatcggcatttccctgatctctggcatcattgacaccgccaaggatatctactatctgttttctgggcaggagaagcctgtcgacccagtgatcaaattctttaacacatacgctggactggtcagcgattcaagcaagatgggcgtgagaaaatgcctgactcccggggaagacaccctgatctatattgcatacaagaatgattcctctttcaaacagaacaccgaggcaatggccctgtactttctggacgtgatcaactccgagattctgtatctgaatacctctaacctggtcctggaataccacctgaaggtggcttgtccaatcggcacactgaggagcgtcgatgtggacatcactgcctataccattctgtacgataccgctgacaacatcaagaaatacaagttcattcgcatggccacactgctgtccaaacatcccgtgatccgactgacatgcggactggcagctactctggtcatcaagccttacgaggtgccaattagcgacatgcagctgctgaagatggcaaccccaggagagcctgaaagtacaaaatcaatccccagcgacgtgtgcgaccgctatcctctgaagaaattctacctgctggcaggaggatgcccatacgacacaagtcagacttttatcgtgcacaccacatgtagcattctgctgcgcactgccacctgggatcagttccggaatagatgggtgctgcagaacccttttcggcaggagggcacctataagcagctgttcacattttccaagtacgatttcaatgacactatcattgaccccaacggggtggctggacatgcatccttctgcacaaaccggagttcaaatcagtgtttttggtctgagcctatgatcctggaagatgtcagctcctgctctagtaggacccgcaagatctatgtgaaactgggcatcttcaacacagaagggttcaattcttttgtgctgaactgtcctactggcagtacaccaacttacatcaaggacaaaaacaccgatagcaataacgtcatcattgagctgcctgtgggcgactatgggactgccaagctgtactccgtcaccaaaccatctagaatcgccgtgttctgcacccacaattatgacaagaggtttaaaagcgatatcattgtgctgatcttcaacagtatctcaggcattcctttttcaagcatctacacaggctccgtgaatgggcgaaaccggctgttcactaccctgagcaaggggatgccatatcggtccatgtactgcgacaatcgccgaccagggtgttactatgccggaatcccctttaatgagaactccgtggaatctgatctgcactatggccccgagattatgctgaaggaaacctacgacacaaactcaatcgatcctcaggtcatcacaaagagcaaaactcatttccctaccccaatctccgtcaagtttaccgtggacaatctggggaacggatataacaagccagagaatttctggaaggatgcaaaatctaagaaaagaacatacagtgccatgactatcaagattctgccctgcaccgtcaggaacaagaacgtggacttcggatataattacggccacatcatttccaacatggtgtacgcccagagcacatcccaggattatggcgacgggactaattacacctttaagagcgtgaacagatccgaccatgagtgcgaaagcatcctggatctgaaggctaaagaggtcaccgtgatgtgccccgcattctcaatccctaggaacattagcgcctacgaaggcctgtgcttttctgtgacaactagtaaggaccactgtgcttctaataaggagtggctgaaaagttatggatacggcaacacagatgcaactaaacagagagtctgcttccaccattggaattatgtgaccacatccctggactactattgttcttacgaagacatctggaagtccgattggcccgattacgacccttgtaagtcttacatctacatcgagtacagggatatctggattgaatccaaggtgctgcagcagcccccttatacattcgagtttactcatgacgattctaacgagtacgtgaataaggaaatcagtaacaaactgaatgacctgtacaacgagtacaagaacatcatggaatacagtgatggctcactgcctgcttctattaaccgcctggcaaaaagcctgacctccgagggacgagaaattgccagcgtgaacattgacggcaatctgctggatatcgcctaccaggctgacaaggaaaaaatggctgatattcagaacaagatcaatgacattacacgcgatctgttcatccacactctgagcaataaggatatcaaggacatcattgagtccgaggaagggaagcggtgctgtatcattgacgtgaagaacaacagagtgaagaaatactacccaatcgataactacctgtgcggaaccctggacgattacatctacacaagtgtcgagtacaataagtcatacgtgctgatcaacgacacctatatgagctatgattacctggagtcctctggcgtggtcgtgctgagttgttacgaaatgactatcatttcactggataccaaggacgcaaaagatgccatcgaggacgaaattgtcgcttccgcagtggccgaggctctgaatgacatgttcaaggaattcgataagaacgtctctgtgatcattatcaaggaggaagataactatctgaatagttcacccaacatctatcatatcatctacatcatcggaggcaccatcctgattctgctggtcattatcctgatcctggtcatctacattgcctgcaacaagtatagaaccaggaagtacaaaatcatgaaggacgatacaatgagcatcaagagtgagcaccataatagtctggagaccgtgtcaatggaaatcatggacaacagatattaa

SEQ ID NO: 11 - DNA, 31 bp, Artificial Sequence
  Primer
gtagacctgtagccgtctgtgcttaatagag

SEQ ID NO: 12 - DNA, 59 bp, Artificial Sequence
  Primer
atccatatgactagtagatcctctagagggagtcgaatatggtgtaaatcacaattgat

SEQ ID NO: 13 - DNA, 59 bp, Artificial Sequence
  Primer
atcaattgtgatttacaccatattcgactccctctagaggatctactagtcatatggat

SEQ ID NO: 14 - DNA, 62 bp, Artificial Sequence
  Primer
ctggaagaagataaacatcaccataaacaatgtagaaatctagagcggatccgcaggtttgc

SEQ ID NO: 15 - DNA, 62 bp, Artificial Sequence
  Primer
gcaaacctgcggatccgctctagatttctacattgtttatggtgatgtttatcttcttccag

SEQ ID NO: 16 - DNA, 25 bp, Artificial Sequence
  Primer
aactaacgcctacgcctaaacccgc

SEQ ID NO: 17 - DNA, 32 bp, Artificial Sequence
  Primer
atcggatgatgatatcactgtttccagagtag

SEQ ID NO: 18 - DNA, 86 bp, Artificial Sequence
  Primer
atccatatgactagtagatcctctagatattgaatatataaacctttttacattcattatattataattacttatagtacttcaag

SEQ ID NO: 19 - DNA, 86 bp, Artificial Sequence
  Primer
cttgaagtactataagtaattataatataatgaatgtaaaaaggtttatatattcaatatctagaggatctactagtcatatggat

SEQ ID NO: 20 - DNA, 61 bp, Artificial Sequence
  Primer
gctaacgtaataatgcgttatgaagacacttatatcatctagagcggatccgcaggtttgc

SEQ ID NO: 21 - DNA, 61 bp, Artificial Sequence
  Primer
gcaaacctgcggatccgctctagatgatataagtgtcttcataacgcattattacgttagc

SEQ ID NO: 22 - DNA, 30 bp, Artificial Sequence
  Primer
agaggagatcaagggtttggatcaacagga

SEQ ID NO: 23 - DNA, 29 bp, Artificial Sequence
  Primer
acgttgttatgcgtactacctgctgttgt

SEQ ID NO: 24 - DNA, 63 bp, Artificial Sequence
  Primer
atccatatgactagtagatcctctagagtcacaggaacaaactaatactataatggagattag

SEQ ID NO: 25 - DNA, 63 bp, Artificial Sequence
  Primer
ctaatctccattatagtattagtttgttcctgtgactctagaggatctactagtcatatggat

SEQ ID NO: 26 - DNA, 70 bp, Artificial Sequence
  Primer
acaactcaaattacgatttcaatatataatcttgatgtaattagtgtctagagcggatccgcaggtttgc

SEQ ID NO: 27 - DNA, 70 bp, Artificial Sequence
  Primer
gcaaacctgcggatccgctctagacactaattacatcaagattatatattgaaatcgtaatttgagttgt

SEQ ID NO: 28 - DNA, 25 bp, Artificial Sequence
  Primer
taatcagtgttgggtacgaccgcct

SEQ ID NO: 29 - DNA, 24 bp, Artificial Sequence
  Primer
ggacgtacaccacttcattgcgct

SEQ ID NO: 30 - DNA, 68 bp, Artificial Sequence
  Primer
atccatatgactagtagatcctctagagattgctaatgttacgtatatcattttcgatatccatgatg

SEQ ID NO: 31 - DNA, 68 bp, Artificial Sequence
  Primer
catcatggatatcgaaaatgatatacgtaacattagcaatctctagaggatctactagtcatatggat

SEQ ID NO: 32 - DNA, 58 bp, Artificial Sequence
  Primer
acgagtaatgaactgaaattacagtaccaaactgtctagagcggatccgcaggtttgc

SEQ ID NO: 33 - DNA, 58 bp, Artificial Sequence
  Primer
gcaaacctgcggatccgctctagacagtttggtactgtaatttcagttcattactcgt

SEQ ID NO: 34 - DNA, 26 bp, Artificial Sequence
  Primer
tgataagcgacgccattcatgtcgga

SEQ ID NO: 35 - DNA, 31 bp, Artificial Sequence
  Primer
atcgaggagactgtctagaagccgtttatgt

SEQ ID NO: 36 - DNA, 51 bp, Artificial Sequence
  Primer
atccatatgactagtagatcctctagagattgccggtcacaaacaagcccg

SEQ ID NO: 37 - DNA, 51 bp, Artificial Sequence
  Primer
cgggcttgtttgtgaccggcaatctctagaggatctactagtcatatggat

SEQ ID NO: 38 - DNA, 58 bp, Artificial Sequence; identical to SEQ ID NO: 32
  Primer

SEQ ID NO: 39 - DNA, 58 bp, Artificial Sequence; identical to SEQ ID NO: 33
  Primer

SEQ ID NO: 40 - DNA, 25 bp, Artificial Sequence
  Primer
acttcgccgtgggtgttagttgtct

SEQ ID NO: 41 - DNA, 31 bp, Artificial Sequence
  Primer
cgtgcgcaattagatctaaagaagatgttcc

SEQ ID NO: 42 - DNA, 52 bp, Artificial Sequence
  Primer
tgactagtagatcctctagacttaaaaaatggttagagccaagggcgttaac

SEQ ID NO: 43 - DNA, 52 bp, Artificial Sequence
  Primer
gttaacgcccttggctctaaccattttttaagtctagaggatctactagtca

SEQ ID NO: 44 - DNA, 62 bp, Artificial Sequence
  Primer
gatttttctagcctaattattataaaaagtattttatatctatctagagcggatccgcaggt

SEQ ID NO: 45 - DNA, 62 bp, Artificial Sequence
  Primer
acctgcggatccgctctagatagatataaaatactttttataataattaggctagaaaaatc

SEQ ID NO: 46 - DNA, 35 bp, Artificial Sequence
  Primer
cctatatatcgcatcatcttgaaagtcacacaatg

SEQ ID NO: 47 - DNA, 29 bp, Artificial Sequence
  Primer
cctggtacctcatgaatttacagagatta

SEQ ID NO: 48 - DNA, 33 bp, Artificial Sequence
  Primer
ccggaattccgcatgccccaattgattgtcatg

SEQ ID NO: 49 - DNA, 32 bp, Artificial Sequence
  Primer
ccggaattcgcgcgcaataaatacagaaccag

SEQ ID NO: 50 - DNA, 34 bp, Artificial Sequence
  Primer
ccgctcgagtaccgattatccataatttccatag

SEQ ID NO: 51 - DNA, 29 bp, Artificial Sequence
  Primer
tcgggatccccatgaatttacagagatta

SEQ ID NO: 52 - DNA, 33 bp, Artificial Sequence
  Primer
tgccccgggctcgagttccgcatgccccaattg

(19) United States
(12) Patent Application Publication
(10) Pub. No.: US 2014/0335115 A1
(43) Pub. Date: Nov. 13, 2014

(54) SUPPRESSORS OF MATURE T CELLS

(71) Applicants: Dina Alzhanova, Beaverton, OR (US); Klaus Frueh, Portland, OR (US); Erika Hammarlund, Hillsboro, OR (US); Mark Slifka, Banks, OR (US)

(72) Inventors: Dina Alzhanova, Beaverton, OR (US); Klaus Frueh, Portland, OR (US); Erika Hammarlund, Hillsboro, OR (US); Mark Slifka, Banks, OR (US)

(73) Assignee: Oregon Health & Science University, Portland, OR (US)

(21) Appl. No.: 14/272,355

(22) Filed: May 7, 2014

Related U.S. Application Data
(60) Provisional application No. 61/820,481, filed on May 7, 2013.

(51) Int. Cl. C07K 14/005; C12N 7/00

(52) U.S. Cl. CPC C07K 14/005; C12N 7/00; C12N 2710/24133; A61K 39/00
USPC 424/186.1; 435/320.1; 514/44 R

(57) ABSTRACT

Disclosed herein is a viral polypeptide and homologs thereof that inhibit an immune response, particularly the response of memory and effector CD4⁺ and CD8⁺ T cells.

### PRIORITY CLAIM

This application claims the benefit of U.S. Provisional Application 61/772,962, filed 5 Mar. 2013 which is incorporated by reference herein.

### ACKNOWLEDGEMENT OF GOVERNMENT SUPPORT

This invention was made with the support of the United States government under the terms of grant numbers OD011092-53 and AI077048-01 awarded by the National Institutes of Health. The United States government has certain rights in this invention.

### FIELD

Generally, the field is pharmaceutical compositions comprising recombinant viral polypeptides. More specifically, the field is immunosuppressive pharmaceutical compositions comprising recombinant viral polypeptides.

### BACKGROUND

The DNA genomes of orthopoxviruses encode approximately 200 open reading frames (ORFs) with around 90 highly conserved genes encoded in the central regions of the genome whereas the terminally coded genes vary among different orthopoxviruses and are responsible for differences in host range, virulence, and immune evasion (Gubser C et al, J Gen Virol 85, 105-117 (2004); incorporated by reference herein). Conserved genes among orthopoxviruses are highly related to each other resulting in cross-protection, i.e. prior infection with any one of the orthopoxviruses generally protects against serious disease by other orthopoxviruses. For example, vaccinia virus (VACV) is broadly protective against all other orthopoxviruses.

Protection against OPXV is remarkably long lived. During a 2003 MPXV outbreak, the number of lesions in previously vaccinated individuals was significantly lower with some individuals being completely protected from MPXV-associated disease (Hammarlund E et al, Nat Med 11, 1005-1011 (2005); incorporated by reference herein). Antibody (Ab) titers to the vaccine remain remarkably stable over the life of vaccinated individuals (Hammarlund E et al, Nat Med 9, 1131-1137 (2003); incorporated by reference herein) and vaccine-mediated protection of non-human primates (NHP) against lethal MPXV challenge is antibody mediated (Edghill-Smith Y et al, Nat Med 11, 740-747 (2005); incorporated by reference herein). Similarly, vaccinated mice succumb to lethal challenge with mousepox ectromelia virus (ECTV) in the absence of antibody, despite the presence of poxvirus-specific T cells (Panchanathan V et al, J Virol 80, 6333-6338 (2006); incorporated by reference herein.) In contrast, T cells promote survival of vaccinated mice challenged with lethal doses of vaccinia virus (Belyakov I M et al, Proc Natl Acad Sci USA 100, 9458-9463 (2003) and Snyder J T et al, J Virol 78, 7052-7060; both of which incorporated by reference herein).

The limited role of T cells in protecting against virulent orthopoxviruses is surprising given that orthopoxviruses induce a strong T cell response recognizing multiple conserved epitopes (Tscharke D C et al, J Exp Med 201, 95-104 (2005); incorporated by reference herein). Moreover, vaccinia virus is widely used as a vaccine vector that induces a T cell response (Grandpre L E et al, Vaccine 27, 1549-1556 (2009) and Earl P L et al, Virology 366, 84-97 (2007); both of which are incorporated by reference herein). Some orthopoxviruses express proteins that allow the virus to evade T cell responses. This has been shown in cowpox virus in which the deletion of two gene products resulted in a more robust T cell response and a less virulent virus (Byun M et al, Cell Host Microbe 6, 422-432 (2009); incorporated by reference herein). Thus, the inability of T cells in protecting against virulent orthopoxviruses might be due to T cell evasion mechanisms.

In the case of cowpoxvirus, T cell evasion is mediated by two gene products that each interfere with different steps of the MHC-I antigen presentation pathway. CPXV203 binds to and retains MHC-I in the endoplasmic reticulum (ER) (Byun M et al, Cell Host Microbe 2, 306-315 (2007); incorporated by reference herein. CPXV12 inhibits TAP-dependent peptide translocation across the ER membrane Byun M et al 2009, supra and Alzhanova D et al, Cell Host Microbe 6, 433-445 (2009) incorporated by reference herein.)

### SUMMARY

Disclosed herein are recombinant nucleic acid expression vectors that encode and express the polypeptide listed herein as SEQ ID NO: 1 or a homolog thereof (examples of such homologs include: SEQ ID NO: 2; SEQ ID NO: 3, SEQ ID NO: 4, SEQ ID NO: 5, SEQ ID NO: 6, SEQ ID NO: 7, and SEQ ID NO: 8 and any mutant form of SEQ ID NO: 1 found to function to suppress effector and memory subsets of CD4⁺ and CD8⁺ T cells). Expression of SEQ ID NO: 1 and homologs thereof by viral vectors such as adenoviral vectors results in the inhibition of CD4+ and CD8+ memory and effector cells.

Disclosed herein are methods of inhibiting CD4⁺ or CD8⁺ T cells in a subject. Such methods involve the administration of an effective amount of an expression vector comprising SEQ ID NO: 1 or a homolog thereof to cells of a subject. The administration can be in vivo administration to cells of the subject including systemic and/or local administration through, for example, injection. The administration can be ex vivo administration to cells removed from a subject then added back to the subject.

Disclosed herein are vaccines that comprise a deleterious mutation in the polypeptide listed herein as SEQ ID NO: 1 and homologs thereof.

Disclosed herein are methods of immunizing a subject against a poxvirus infection comprising immunizing a subject with a vaccine with a deleterious mutation in SEQ ID NO: 1 or a homolog thereof.

### BRIEF DESCRIPTION OF THE DRAWINGS

Some of the drawings in this disclosure are photographic images that may not reproduce properly in a patent application publication. Additionally, some of the drawings may be better understood when viewed in color, which is not available in a patent application publication. Applicants consider all photographic images and color drawings as part of the original disclosure and reserve the right to present high quality and/or color images of the herein described figures in later proceedings.

FIG. 1 is a flow chart describing the PBMC T cell assay used in the Examples below.

FIG. 2 is a flow chart describing the monkey CM9-specific T cell assay used in the Examples below.

FIG. 3A is a set of two bar graphs depicting the number of activated (IFNγ⁺/TNFα⁺) CD4 (black) and CD8 (gray) T cells produced after infection of PBMC with the indicated constructs using the assay described in FIG. 1 as a percentage of those produced upon infection with vaccinia virus (left panel) and as a percentage of those produced by uninfected cells. The left panel shows MHC dependent responses and the right panel shows MHC independent responses.

FIG. 3B is a map showing deletions in the MPXV-US2003 described further herein.

FIG. 3C is a bar graph depicting the number of activated (IFNγ⁺/TNFα⁺) CD4 (black) and CD8 (gray) T cells produced after infection of PBMC with the indicated constructs using the assay described in FIG. 1 as a percentage of those produced upon infection with vaccinia virus.

FIG. 3D is a set of four flow cytometry plots depicting the number of activated (IFNγ⁺/TNFα⁺) T cells produced using the assay described in FIG. 2 using the MPXV US2003 Δ197 mutant and wild type viruses. Top panels and bottom panels represent different animals.

FIG. 4A is a set of two bar graphs depicting inhibition of two separate T cell clones by SEQ ID NO: 1 in terms of IFNγ spot forming units determined by ELISPOT.

FIG. 4B is a set of flow cytometry plots depicting inhibition of CM9-specific CD8 T cell responses by SEQ ID NO: 1 in terms of decreased numbers of IFNγ⁺TNFα⁺ T cells by the assay described in FIG. 2 above.

For FIGS. 5A, 5B, 5C, and 5D, 8 rhesus macaques were inoculated intrabronchially with MPXVUS2003 or MPXV Δ197 at day 0 post infection. PBMC were purified on whole blood on the indicated number of days post infection.

FIG. 5A is a bar graph depicting the results of activation of IFNγ⁺TNFα⁺CD4⁺ T cells produced in response to MHC dependent stimulation in the presence of MPXV US2003 Δ197 mutant and wild type viruses. Assay was performed as described

FIG. 5B is a bar graph depicting the results of activation of IFNγ⁺TNFα⁺CD8⁺ T cells produced in response to MHC dependent stimulation in the presence of MPXV US2003 Δ197 mutant and wild type viruses.

FIG. 5C is a line graph depicting the results of PBMC CD4+ and CD8+ T cell responses to MHC independent stimulation with wild type or MPXVΔ197 infected animals.

FIG. 5D is a line graph depicting the mean percentage of CD4⁺ and CD8⁺ in PBMC for wild type and MPXVΔ197 infected animals by intracellular cytokine staining.

FIG. 6A is a set of two bar graphs summarizing the results of experiments performed as follows: Left Panel—PBMC from VACV-immune subjects (n=4) were infected with VACV, MPXV Zaire or MPXV US2003 (MOI of 0.5) for 18 h. Poxvirus-specific CD4+ and CD8+ T cell responses were measured by ICCS. Results are normalized to % of VACV-specific response. Right Panel—PBMC from VACV-naïve subjects (n=3) were infected with indicated viruses or uninfected (UN) and T cells were stimulated with plate-bound αCD3 Ab for 6 h.

FIG. 6B is a bar graph summarizing the results when CM9-specific RM CD8+ T-cells were incubated with HFF infected with MPXV US2003 (MOI of 2) in the presence or absence of 10 μM ST246 for 18 h prior to stimulation with CM9-peptide pulsed BLCL cells for 6.5 h. The percentage of IFNγ⁺TNFα⁺ cells as measured by ICCS is shown.

FIG. 6C is a map of 10 Kb deletions (light grey) or a single ORF 197 deletion (black) in the terminal regions of the MPXV US2003 genome (black).

FIG. 6D is a bar graph summarizing the results when human CD4⁺ and CD8⁺ T cell responses to MPXV deletion mutants were determined by ICCS as in FIG. 6A. Infection rates of CD14⁺ monocytes in PBMC for MPXV US2003, MPXVΔ184-193, MPXVΔ194-197, and MPXVΔ197 were 73%, 81%, 65%, and 70%, respectively.

FIG. 6E is a bar graph summarizing the results when Inhibition of CM9-specific CD8⁺ T-cell stimulation by MPXV US2003 or deletion mutants was measured by ICCS as in FIG. 6B. T cells were co-incubated with HFF cells infected with indicated viruses (MOI of 2, 10 μM ST246) for 18 h and then stimulated with CM9-peptide pulsed BLCLs for 6.5 h.

FIG. 7A is a Schematic representation of MPXV 197 with predicted signal peptide (SP, blue, SignalP), transmembrane domains (TM, green, TMP, red), and N-linked glycosylation sites (red, NetNGlyc 1.0).

FIG. 7B is a set of two images of Western blots of CHO cells transduced with Ad-197/Ad-tTA (‘Ad-197’) or Ad-tTA only (‘Ad-control’) for 24 hours and lysed in sample buffer prior to electrophoretic separation and immunoblotting with αFLAG. Right panel is the same Western blot overexposed. Overexposure reveals a >250 kDa band (asterisk).

FIG. 7C is set of images of a Western blot of CHO cells transduced as in FIG. 7B. After 24 h, cell surface proteins were biotinylated followed by immunoprecipitation with NeutrAvidin, electrophoretic separation and immunoblotting with αFLAG.

FIG. 7D is a set of images of Western blots resulting from the following—24 h after transduction with the indicated expression vectors, CHO cells were metabolically labeled for 45 min followed by chase for 0.5, 1, and 3 h. Cell lysates were immunoprecipitated with αFLAG. In the right panel, samples were treated with EndoH or left untreated prior to electrophoretic separation. EndoH sensitive proteins are indicated by asterisks.

FIG. 7E is a set of fluorescent images showing sub-cellular localization of C- and N-terminal FLAG fusions of MPXV197 was determined by IFA using αFLAG. CHO cells were either permeabilized (‘Intracellular’) or non-permeabilized (‘Cell-surface’) prior to IFA. Scale bar is 20 μm. Arrows indicate the plasma membrane.

FIG. 8A is bar graph summarizing the results of CM9-specific CD8+ T-cells were incubated (18 h) with untreated CHO cells (UN) or CHO cells transduced with either Ad-197/Ad-tTA (‘Ad-197’) or Ad-tTA only (‘Ad-control’) and stimulated with CM9-peptide pulsed BLCLs. The percentage of INFγ⁺ TNFα⁺ CD8⁺ T-cells was determined by ICCS.

FIG. 8B is a line graph showing the kinetics of T cell inhibition by MPXV197 CM9-specific T-cells. Cells were incubated with Ad-197/Ad-tTA or Ad-tTA-transduced CHO cells for indicated time periods, washed, and stimulated with peptide pulsed BLCLs.

FIG. 8C is a set of two bar graphs summarizing the results when human Mtb specific CD8+ T cell clones D466 D6 and D160 1-23 were stimulated with BEAS-2b cells transduced with Ad-197/Ad-tTA or Ad-tTA only in the presence of CFP10₂₋₁₂ peptide or pronase digested Mtb cell wall, respectively. For MHC-independent stimulation, both clones were incubated with PHA. The number of IFNγ+ T cells was measured by ELISPOT.

FIG. 8D is a set of two bar graphs summarizing the results when CM9-specific CD8+ T cells were incubated (18 h) with CHO cells transduced with Ad-197/Ad-tTA or Ad-tTA, washed, and stimulated either with PMA/lonomycin or CM9-peptide pulsed BLCLs. Left panel: The percentages of INFγ⁺ TNFα⁺ T-cells were determined by ICCS with stimulation in the presence of uninfected CHO cells set to 100% (MAX). Right Panel: The percent live CD8+ T cells was determined by LIVE/DEAD Fixable Dead Cell Stain.

FIG. 8E is a set of three flow cytometry histograms of MaMu-A*01/CM9 tetramer staining of CM9-specific CD8+ T cells after 18 h of incubation with MPXV197-expressing CHO cells (‘Ad-197’) or control cells (‘Ad-control’).

FIG. 9A is a sequence comparison of MPXV 197 and selected homologs by Geneious v5.6.3. Black, green, and red bars show consensus sequence, conserved, and hydrophobic residues, respectively.

FIG. 9B is an image of Western blots of CHO cells transduced with Ad-B22R, Ad-197 and Ad-tTA for 24 h followed by immunoblotting with αFLAG. Right panel: Overexposure reveals a >250 kDa band (asterisk).

FIG. 9C is an image of a Western blot CHO cells transduced as in FIG. 9B. After 24 h, cell surface proteins were biotinylated followed by immunoprecipitation with NeutrAvidin, electrophoretic separation and immunoblotting with αFLAG.

FIG. 9D is a set of fluorescent images of CHO cells transfected with pCDNA3.1-B22-CFlag (24 h), fixed, and either permeabilized (‘intracellular’) or left unpermeabilized (‘cell-surface’). The samples were stained with αFLAG and analyzed by LSCM. The scale bar is 20 μm.

FIG. 9E is a bar graph summarizing the results of BEAS-2b cells, uninfected (UN) or transduced with Ad-197/Ad-tTA (‘Ad-197’) or Ad-tTA only (‘Ad-control’) were used to stimulate human Mtb-specific T cell clone D466 D6 with CFP10₂₋₁₂ peptide.

FIG. 9F is a bar graph summarizing the results of CM9-specific T-cells incubated (18 h) with CHO cells either uninfected (UN) or transduced with Ad-B22R/Ad-tTA (‘Ad-B22R’) or Ad-tTA only (‘Ad-control’) followed by stimulation with CM9-peptide pulsed BLCLs.

FIG. 10A is a bar graph summarizing the results of Human Mtb-specific T cell clone D466 D6 incubated with BEAS-2b cells uninfected (UN) or infected with VACV or VACV-219 (3 h) prior to addition of CFP10₂₋₁₂ peptide. The number of IFNγ⁺ T cells was determined by ELISPOT.

FIG. 10B is a bar graph summarizing the results of CM9-specific T-cells incubated with HFF infected with VACV or VACV-219 for 18 h and stimulated with CM9-peptide pulsed BLCLs. The percentage of INFγ⁺ TNFα⁺ CD8⁺ T cells was determined by ICCS.

FIG. 10C is a bar graph summarizing the results of PBMC from VACV-immune subjects (n=3) infected with indicated viruses (optimized MOI of 0.3-0.6) for 18 h. The infection rates for CD14⁺ cells were VACV (54%), CPXV (45%), CPXV Δ12Δ203-221 (51%), CPXVΔ12-203 (60%), CPXVΔ11-38 (72%), and CPXVΔ204-221 (73%). The percentage of CD4+ and CD8+ responding to poxvirus infection was determined by ICCS for IFNγ and TNFα. The frequency of VACV-reactive T cells was set to 100%.

FIG. 10D is a bar graph summarizing the results of splenocytes from VACV-immunized mice incubated with A20 cells infected with indicated viruses (MOI 5.0) for 6 h. The frequency of poxvirus-reactive T cells was determined by ICCS for IFNγ and TNFα relative to the frequency of VACV-reactive T cells which was set to 100%.

FIG. 10E is an image of a Western blot of Splenocytes from VACV-immunized mice were incubated with A20 cells infected with indicated viruses (MOI 5.0) for 6 h. The frequency of poxvirus-reactive T cells was determined by ICCS for IFNγ and TNFα relative to the frequency of VACV-reactive T cells which was set to 100%.

FIG. 10F is an image of a set of Western blots of CHO cells infected with CPXV, CPXVΔ219 (MOI=5.0) or uninfected (UN) using αCPXV219 Ab. Right Panel: Immunoblot with αCPXV219 Ab of CHO cells infected with VACV, VACV-219 (MOI=5.0) or uninfected (UN), or co-infected with T7-polymerase expressing VACV VTF7-3 (MOI=5.0).

FIG. 11A is an immunization schedule: 4 female RM were inoculated intrabronchially with 2×10⁵ PFU of MPXV US2003 (WT) or MPXVΔ197 on day 0. Whole blood, BAL, and PBMC samples were taken on indicated dpi. 2 RM infected with MPXVUS2003, WT-4 and WT-3, were euthanized at 12 and 24 dpi, respectively. The remaining WT-infected were euthanized on days 37 and 38 pi. Animals infected with MPXVΔ197 were euthanized at 41 and 42 dpi.

FIG. 11B is a plot showing the Average nighttime body temperature (7 PM to 7 AM) as determined by biotelemetry transmitters for RM infected with WT (black) or MPXVΔ197 (red) (mean+/−SEM). P=0.0007 (area under curve (AUC), F-test).

FIG. 11C is a plot of viral loads determined by qPCR in BAL.

FIG. 11D is a plot of viral loads determined by qPCR in whole blood.

FIG. 11E is a plot of the number of skin lesions in WT (blue) or MPXVΔ197 (red)-infected RM. The p-value for the AUC comparison is P=0.0003 (F-test).

FIG. 11F is a plot of poxvirus-specific antibody titers determined by ELISA using VACV as antigen. The titers were not statistically different between WT and MPXVΔ197 cohorts.

FIG. 12A is a set of two plots summarizing the results of PBMC from WT (blue) and MPXVΔ197 (red)-infected RM were infected with VACV (MOI of 0.3) for 18 h. The background-subtracted frequency of poxvirus-responsive CD4+ and CD8+ T cells was determined by ICCS for TNFα and IFNγ. The differences were statistically significant at day 21 (P=0.0063, F-test) for CD4+ T cells and at day 14 (P=0.0069, F-test) for CD8+ T cells.

FIG. 12B is a set of two plots of the total frequency of CD4+ and CD8+ relative to day 0 as determined by flow cytometry. The frequencies were not statistically different between WT and MPXVΔ197 cohorts.

FIG. 12C is a set of two plots of the percentage of CD4⁺ and CD8⁺ T cells relative to day 0 responding to anti-CD3 stimulation determined by ICCS for IFNγ and TNFα. PBMC from WT (blue) or MPXVΔ197 (red) infected animals were stimulated with plate-bound αCD3 Ab for 6 h. The differences were statistically significant at day 14 (P=0.0065, two-tailed t-test) for CD8+ T cells.

FIG. 13 is a set of two bar graphs showing the results of HFF cells infected with indicated viruses (MOI=2) were layered with Jurkat T cells at 24 h post infection After overnight co-incubation, Jurkat T cells were removed, washed, transferred into a fresh plate, and incubated for additional 24 h. The number of infected GFP+ cells was measured by flow cytometry.

FIG. 14A is an illustration of MPXV US2003 recombinant deletion mutant viruses generated by in-vivo recombination replacing ORFs of interest by an expression cassette for eGFP and GPT.

FIG. 14B is a plot showing Multi-step growth kinetics of MPXV-US2003 and MPXVΔ197. BSC40 cells were infected with indicated viruses at 0.1 MOI. After 30 min of incubation, the inoculum was replaced with growth medium. The cells were incubated for indicated time points, harvested, and used for virus titering.

FIGS. 15A and 15B are plots showing the SNP frequency (>0.5%) compared to a reference sequence. Top: MPXV US2003 compared to US2003-39 sequence in public database (GenBank accession # DQ11157). Bottom: MPXVΔ197 mutant virus compared to predicted sequence.

### SEQUENCE LISTING

SEQ ID NO: 1 is ORF197 from monkeypox strain US2003-039 (GenBank AAY9788).

SEQ ID NO: 2 is the homolog from monkeypox strain Copenhagen 58 (GenBank AAX09272).

SEQ ID NO: 3 is the homolog from monkeypox strain Zaire-1979-005 (GenBank AAY97391).

SEQ ID NO: 4 is the homolog from Variola variola major strain Bangladesh 1975 (Genbank AAA60931).

SEQ ID NO: 5 is the homolog from camelpox strain CMS (GenBank AAG37713).

SEQ ID NO: 6 is the homolog from cowpox strain Brighton Red (Genbank NP619999).

SEQ ID NO: 7 is the homolog from Ectromelia strain Moscow (Genbank NP671688).

SEQ ID NO: 8 is the homolog from Molluscum contagiosum virus subtype 1 (Genbank NP043986).

SEQ ID NO: 9 is a codon optimized nucleic acid sequence of MPX197.

SEQ ID NO: 10 is a codon optimized nucleic acid sequence of Variola virus B22R

SEQ ID NO: 11 is the sequence of primer MPXVus8580n-F.

SEQ ID NO: 12 is the sequence of primer MPXVus9760-GFP-R.

SEQ ID NO: 13 is the sequence of primer MPXVus9760-GFP-F.

SEQ ID NO: 14 is the sequence of primer MPXVus9760-Gpt-R.

SEQ ID NO: 15 is the sequence of primer MPVus9760-Gpt-F.

SEQ ID NO: 16 is the sequence of primer MPXVus20700R.

SEQ ID NO: 17 is the sequence of primer MPXVus20288n-F.

SEQ ID NO: 18 is the sequence of primer MPXVus21233-GFP-R.

SEQ ID NO: 19 is the sequence of primer MPXVus21233-GFP-F.

SEQ ID NO: 20 is the sequence of primer MPXVus30468-Gpt-R.

SEQ ID NO: 21 is the sequence of primer MPXVus30468-Gpt-F.

SEQ ID NO: 22 is the sequence of primer MPXVus31330-R.

SEQ ID NO: 23 is the sequence of primer MPXVus167080-F.

SEQ ID NO: 24 is the sequence of primer MPXVus168084-GFP-R.

SEQ ID NO: 25 is the sequence of primer MPXVus168084-GFP-F.

SEQ ID NO: 26 is the sequence of primer MPXVus179413-Gpt-R.

SEQ ID NO: 27 is the sequence of primer MPXVus179413-Gpt-F.

SEQ ID NO: 28 is the sequence of primer MPXVus179957-R.

SEQ ID NO: 29 is the sequence of primer MPXVus178592n2-F.

SEQ ID NO: 30 is the sequence of primer MPXVus179559-GFP-R.

SEQ ID NO: 31 is the sequence of primer MPXVus179559-GFP-F.

SEQ ID NO: 32 is the sequence of primer MPXVus188458-Gpt-R.

SEQ ID NO: 33 is the sequence of primer MPXVus188458-Gpt-F.

SEQ ID NO: 34 is the sequence of primer MPXVus188670-R.

SEQ ID NO: 35 is the sequence of primer MPVus182428-F.

SEQ ID NO: 36 is the sequence of primer MPVusD197-GFP-R.

SEQ ID NO: 37 is the sequence of primer MPVusD197-GFP-F.

SEQ ID NO: 38 is the sequence of primer MPXVus188458-Gpt-R.

SEQ ID NO: 39 is the sequence of primer MPXVus188458-Gpt-F.

SEQ ID NO: 40 is the sequence of primer MPVus189027-R.

SEQ ID NO: 41 is the sequence of primer MPV184-250U-F.

SEQ ID NO: 42 is the sequence of primer MPV184U-5GFP-R.

SEQ ID NO: 43 is the sequence of primer 5GFP-MPV184U-F.

SEQ ID NO: 44 is the sequence of primer 3GPT-MPV184-R.

SEQ ID NO: 45 is the sequence of primer MPV184D-3GPT-F.

SEQ ID NO: 46 is the sequence of primer MPV184-250D-R.

SEQ ID NO: 47 is the sequence of primer NcoI-219-5′-SphI-F.

SEQ ID NO: 48 is the sequence of primer NcoI-219-5′-SphI-R.

SEQ ID NO: 49 is the sequence of primer BssH1-219-3′-XhoI-F.

SEQ ID NO: 50 is the sequence of primer BssH1-219-3′-XhoI-R.

SEQ ID NO: 51 is the sequence of primer CPXV219-GST-F.

SEQ ID NO: 52 is the sequence of primer CPXV219-GST-R.

### DETAILED DESCRIPTION

### I. Terms

Administration: To provide or give a subject an agent by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Administration can be directed to cells of a subject including the administration of an expression vector that results in one or more cells expressing an exogenous protein such as SEQ ID NO: 1 or a homolog thereof. Administration to the cells of the subject can be in vivo (through any route of administration described above) or ex vivo.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. T cell epitopes are formed from contiguous amino acids.

Antigen presenting cell (APC): A cell that can present an antigen to a T cell such that the T cells are activated. The major function of APCs is to obtain antigen in tissues, migrate to lymphoid organs and present the antigen in order to activate T cells. When an appropriate maturational cue is received, both the T cells and APCs are signaled to undergo rapid morphological and physiological changes that facilitate the initiation and development of immune responses. Among these are the up-regulation of molecules involved in antigen presentation including cytokines such as TNFα and IFNγ.

CD4: Cluster of differentiation factor 4, a T cell surface protein that mediates interaction with the MHC Class II molecule. Cells that express CD4 are often helper T cells.

CD8: Cluster of differentiation factor S, a T cell surface protein that mediates interaction with the MHC Class I molecule. Cells that express CD8 are often cytotoxic T cells.

Conservative variants: A substitution of an amino acid residue for another amino acid residue having similar biochemical properties. “Conservative” amino acid substitutions are those substitutions that do not substantially affect or decrease an activity or antigenicity of the Mycobacterium polypeptide. A peptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples.

| Original Amino | Conservative |
|---|---|
| Acid | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; lie |
| Phe | Met; Leu; Tyr |
| Ser | Thr |
| Thr | Ser |
| Trp | Tyr |
| Tyr | Trp; Phe |
| Arg | Lys |
| Asn | Gln, His |
| Val | Ile; Leu |

Contacting: refers to placement in direct physical association, including both a solid and liquid form. Contacting can occur in vitro with isolated cells or tissue or in vivo by administering to a subject.

Cytokine: Proteins made by cells that affect the behavior of other cells. In some examples, a cytokine is a chemokine, a molecule that affects cellular trafficking. Specific, non-limiting examples of cytokines include the interleukins (IL-2, IL-4, IL-6, IL-10, IL-21, etc.), tumor necrosis factor (TNF)α and interferon (IFN)γ.

Degenerate variant: A polynucleotide encoding SEQ ID NO: 1 or any homolog thereof that includes a sequence that is degenerate as a result of the genetic code. There are 20 natural amino acids, most of which are specified by more than one codon. Therefore, all degenerate nucleotide sequences are included in this disclosure as long as the amino acid sequence of SEQ ID NO: 1 or any homolog thereof encoded by the nucleotide sequence is unchanged.

Effective amount: refers to an amount of therapeutic agent that is sufficient to generate a desired response, such as reduce or eliminate a sign or symptom of a condition or disease, such as an autoimmune disease like graft-versus-host disease. When administered to a subject, a dosage will generally be used that will achieve target tissue concentrations) that has been shown to achieve in vitro inhibition T cell activation. In some examples, an “effective amount” is one that treats (including prophylaxis) one or more symptoms and/or underlying causes of any of a disorder or disease. In other examples, an effective amount is an amount that prevents one or more signs or symptoms of a particular disease or condition from developing, such as one or more signs or symptoms associated with activation of memory or effector CD4 or CD8 T cells.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (e.g., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included (see e.g., Bitter et al., Meth. Enzymol. 153:516-544, 1987).

When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Functionally Equivalent: Sequence alterations, such as in an epitope of an antigen, which yield the same results as described herein. Such sequence alterations can include, but are not limited to, conservative substitutions, deletions, mutations, frameshifts, and insertions.

Immune response: A response of a cell of the immune system, such as a B cell, natural killer cell, or a T cell, to a stimulus. In some examples, the response is specific for a particular antigen (an “antigen-specific response”). In another example, an immune response is a T cell response, such as a CD4 or CD8 T cell response. In still further examples the immune response is a response of previously activated, mature, effector or memory T cells.

Inhibiting an immune response: any lessening, reduction in magnitude, or other diminution of any aspect of an immune response in response to treatment with an agent such as SEQ ID NO: 1 or a homolog thereof. Examples include reduced expression of any mRNA or polypeptide associated with an immune response such as antibodies, cytokines, chemokines, costimulatory or differentiation markers, or reduced T, B, or other immune cell activation or proliferation.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, the open reading frames are aligned.

ORF (open reading frame): A series of nucleotide triplets (codons) coding for amino acids without any termination codons. These sequences are usually translatable into a polypeptide.

Polypeptide: Any chain of amino acids, regardless of length or posttranslational modification (such as glycosylation, methylation, ubiquitination, phosphorylation, or the like). In one embodiment, a polypeptide is a protein sequence that has at least 50% or greater identity to SEQ ID NO: 1. Polypeptide” is used interchangeably with peptide or protein, and is used to refer to a polymer of amino acid residues. A “residue” refers to an amino acid or amino acid mimetic incorporated in a polypeptide by an amide bond or amide bond mimetic.

Pharmaceutically acceptable: indicates approval by a regulatory agency of the Federal or a state government or listed in the U.S. Pharmacopeia or other generally recognized pharmacopeia for use in animals, and more particularly in humans.

Pharmaceutically acceptable carriers: The pharmaceutically acceptable carriers useful with the polypeptides and nucleic acids described herein are conventional. Remington's Pharmaceutical Sciences, by E. W. Martin, Mack Publishing Co., Easton, Pa., 19th Edition (1995), describes compositions and formulations suitable for pharmaceutical delivery of the polypeptides or polynucleotides herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: A linear nucleotide sequence. Polypeptide: Any chain of amino acids, regardless of length or posttranslational modification (e.g., glycosylation or phosphorylation). A “peptide” is a chain of amino acids that is less than 100 amino acids in length. In one embodiment, a “peptide” is a portion of a polypeptide, such as about 8-11, 9-12, or about 10, 20, 30, 40, 50, or 100 contiguous amino acids of a polypeptide that is greater than 100 amino acids in length.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. The promoter can be a constitutive or an inducible promoter.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Sequence identity/similarity: The identity/similarity between two or more nucleic acid sequences or two or more amino acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (116671554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15720*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

A heterologous nucleic acid sequence is a sequence that does not share a common origin with a first sequence. For example, an adenoviral vector (or CMV vector, or lentiviral vector, etc.) can be made to comprise a heterologous poxvirus sequence (such as MPX197 or a homolog thereof). Additionally, an expression vector designed to express MPX197 can comprise a heterologous promoter (such as a tetracycline inducible promoter) to drive expression of MPX197.

Subject: A living multicellular vertebrate organism, a category that includes, for example, mammals and birds. A “mammal” includes both human and non-human mammals, such as mice. In some examples, a subject is a patient, such as a patient with a disease characterized by inappropriate activation of CD4⁺ and CD8⁺ effector and memory T cells or a patient at risk of developing a poxvirus infection. In other examples, the subject is a primate—which includes human and nonhuman primates.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Treat: refers to any type of treatment that imparts a benefit to a patient afflicted with a disease, including improvement in the condition of the patient (e.g., in one or more symptoms), delay in the progression of the condition, etc. Similarly, “treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology.

Vector: A nucleic acid molecule as introduced into a host cell, thereby producing a protein encoded by a nucleic acid in a host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in mammalian cells. Vectors can be replicating, nonreplicating, persistent or transient. Vectors also include viral vectors, such as, but not limited to, retrovirus, orthopox, avipox, fowlpox, capripox, suipox, adenovirus, herpes virus, alpha virus, baculovirus, Sindbis virus, lentivirus, and poliovirus vectors.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Polynucleotide Vectors Comprising SEQ ID NO: 1 or Homologs Thereof

Disclosed herein are nucleic acid expression vectors that encode and express the polypeptide listed herein as SEQ ID NO: 1 and homologs thereof. Examples of such homologs include: SEQ ID NO: 2; SEQ ID NO: 3, SEQ ID NO: 4, SEQ ID NO: 5, SEQ ID NO: 6, SEQ ID NO: 7, and SEQ ID NO: 8 and any mutant form of SEQ ID NO: 1 found to function to suppress effector and memory subsets of CD4⁺ and CD8⁺ T cells. Expression of SEQ ID NO: 1 and homologs thereof by viral vectors such as adenoviral vectors results in the inhibition of CD4+ and CD8+ memory and effector cells.

A nucleic acid encoding SEQ ID NO: 1 or a homolog thereof can be cloned or amplified by in vitro methods, such as the polymerase chain reaction (PCR), the ligase chain reaction (LCR), the transcription-based amplification system (TAS), the self-sustained sequence replication system (3SR) and the Qβreplicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding SEQ ID NO: 1 or a homolog thereof include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides of the invention can be ribonucleotides, deoxyribonucleotides, or modified forms of either nucleotide. The term includes single and double forms of DNA.

Viral vectors expressing SEQ ID NO: 1 or a homolog thereof disclosed herein can also be prepared. A number of viral vectors have been constructed, including polyoma, SV40 (Madzak et al., 1992, J. Gen. Viral. 73:15331536), adenovirus (Berkner, 1992, Curr. Top. Microbial. Immunol. 158:39-6; Berliner et al., 1988, BioTechniques 6:616-629; Gorziglia et al., 1992, J. Viral. 66:4407-4412; Quantin et al., 1992, Proc. Natl. Acad. Sci. USA 89:2581-2584; Rosenfeld et al., 1992, Cell 68:143-155; Wilkinson et al., 1992, Nucl. Acids Res. 20:2233-2239; Stratford-Perricaudet et al., 1990, Hum. Gene Ther. 1:241-256), vaccinia virus (Mackett et al., 1992, Biotechnology 24:495-499), adeno-associated virus (Muzyczka, 1992, Curr. Top. Microbial. Immunol. 158:91-123; On et al., 1990, Gene 89:279-282), herpes viruses including HSV and EBV (Margolskee, 1992, Curr. Top. Microbial. Immunol. 158:67-90; Johnson et al., 1992, J. Viral. 66:2952-2965; Fink et al., 1992, Hum. Gene Ther. 3:11-19; Breakfield et al., 1987, Mol. Neurobiol. 1:337-371; Fresse et al., 1990, Biochem. Pharmacal. 40:2189-2199), Sindbis viruses (Herweijer et al., 1995, Hum. Gene Ther. 6:1161-1167; U.S. Pat. Nos. 5,091,309 and 5,217,879), alphaviruses (Schlesinger, 1993, Trends Biotechnol. 11:18-22; Frolov et al., 1996, Proc. Natl. Acad. Sci. USA 93: 11371-11377) and retroviruses of avian (Brandyopadhyay et al., 1984, Mol. Cell Biol. 4:749-754; Petropouplos et al., 1992, J. Viral. 66:3391-3397), murine (Miller, 1992, Curr. Top. Microbial. Immunol 158:1-24; Miller et al., 1985, Mol. Cell Biol. 5:431-437; Sorge et al., 1984, Mol. Cell Biol. 4:1730-1737; Mann et al., 1985, J. Viral. 54:40 1-407), and human origin (Page et al., 1990, J. Viral. 64:5370-5276; Buchschalcher et al., 1992, J. Virol. 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.) In one embodiment, the polynucleotide encoding SEQ ID NO: 1 or a homolog thereof is included in a viral vector. Suitable vectors include andenoviral vectors.

Basic techniques for preparing recombinant DNA viruses containing a heterologous DNA sequence encoding SEQ ID NO: 1 or a homolog thereof are known in the art. Such techniques involve, for example, homologous recombination between the viral DNA sequences flanking the DNA sequence in a donor plasmid and homologous sequences present in the parental virus (Mackett et al., 1982, Proc. Natl. Acad. Sci. USA 79:7415-7419). In particular, recombinant viral vectors such as an adenoviral vector can be used in delivering the gene. The vector can be constructed using methods known in the art. Some such methods include using a unique restriction endonuclease site that is naturally present or artificially inserted in the parental viral vector to insert the heterologous DNA.

Generally, a DNA donor vector contains the following elements: (i) a prokaryotic origin of replication, so that the vector may be amplified in a prokaryotic host; (ii) a gene encoding a marker which allows selection of prokaryotic host cells that contain the vector (e.g., a gene encoding antibiotic resistance); (iii) at least one DNA sequence encoding the SEQ ID NO: 1 or the homolog thereof located adjacent to a transcriptional promoter capable of directing the expression of the sequence; and (iv) DNA sequences homologous to the region of the parent virus genome where the foreign gene(s) will be inserted, flanking the construct of element (iii).

Generally, DNA fragments for construction of the donor vector, including fragments containing transcriptional promoters and fragments containing sequences homologous to the region of the parent virus genome into which foreign DNA sequences are to be inserted, can be obtained from genomic DNA or cloned DNA fragments. The donor plasmids can be mono-, di-, or multivalent (e.g., can contain one or more inserted foreign DNA sequences). The donor vector can contain an additional gene that encodes a marker that will allow identification of recombinant viruses containing inserted foreign DNA. Several types of marker genes can be used to permit the identification and isolation of recombinant viruses. These include genes that encode antibiotic or chemical resistance (e.g., see Spyropoulos et al., 1988, J. Viral. 62:1046; Falkner and Moss, 1988, J. Viral. 62:1849; Franke et al., 1985, Mol. Cell. Biol. 5: 1918), as well as genes such as the E. coli lacZ gene, that permit identification of recombinant viral plaques by colorimetric assay (Panicali et al., 1986, Gene 47:193-199), to say nothing of genes that encode fluorescent or light emitting proteins such as GFP, RFP, luciferase, or any other fluorescent protein.

The DNA gene sequence to be inserted into the virus can be placed into a donor plasmid, such as an E. coli plasmid construct, into which DNA homologous to a section of DNA such as that of the insertion site of the viral vector where the DNA is to be inserted has been inserted. Separately the DNA gene sequence to be inserted is ligated to a promoter. The promoter-gene linkage is positioned in the plasmid construct so that the promoter-gene linkage is flanked on both ends by DNA homologous to a DNA sequence flanking a region of viral DNA that is the desired insertion region. With a parental viral vector, a viral promoter is used. The resulting plasmid construct is then amplified by growth within E. coli bacteria and isolated. Next, the isolated plasmid containing the DNA gene sequence to be inserted is transfected into a cell culture, for example chick embryo fibroblasts, along with the parental virus, for example poxvirus. Recombination between homologous pox DNA in the plasmid and the viral genome respectively results in a recombinant poxvirus modified by the presence of the promoter-gene construct in its genome, at a site that does not affect virus viability.

### III. MPXV197

MPXV197 is the largest gene in the monkeypoxvirus genome. It is predicted to be a transmembrane protein that is a member of the B22 family of proteins that is found in cowpoxvirus, ectromelia virus (mousepox) and variola virus. No homolog is found in the vaccinia virus genome. Deletion of MPXV197 from monkeypox severely attenuates MPXV and prevents lethal disease in rhesus macaques. Even though viral titer was substantially reduced, rhesus macaques infected with MPXV197-deleted virus had a stronger and more rapid T cell response than the wild type monkeypox virus.

### IV. Compositions Comprising Vectors

SEQ ID NO: 1 or any homolog thereof or the corresponding nucleic acid encoding SEQ ID NO: 1 or any homolog thereof can be used to inhibit an immune response in a subject. In several examples, the subject has or is at risk of having a disease characterized by an inappropriate response to memory and/or effector T cells. Thus, in several embodiments, the methods include administering to a subject a therapeutically effective amount of a viral vector expressing SEQ ID NO: 1 or the homolog thereof in order to inhibit an immune response, such as, but not limited to, a memory or effector CD4⁺ or CD8⁺ immune response.

Amounts effective for these uses will depend upon the severity of the disease and the general state of the patient's health. In one example, a therapeutically effective amount of the viral vector is that which provides either subjective relief of a symptom(s) or an objectively identifiable improvement as noted by the clinician or other qualified observer.

One approach to administration of nucleic acids is direct injection of plasmid DNA, such as with a mammalian expression plasmid. As described above, the nucleotide sequence encoding SEQ ID NO: 1 or a homolog thereof can be placed under the control of a promoter to increase expression of the molecule.

Methods of administering a viral vector encoding SEQ ID NO: 1 or a homolog thereof into mammals include, but are not limited to, exposure of cells to the recombinant virus ex vivo, or injection of the composition into the affected tissue or intravenous, subcutaneous, intradermal or intramuscular administration of the virus. Alternatively the recombinant viral vector or combination of recombinant viral vectors may be administered locally in a pharmaceutically acceptable carrier. Generally, the quantity of recombinant viral vector, carrying the nucleic acid sequence encoding SEQ ID NO: 1 or a homolog thereof administered is based on the titer of virus particles. One of skill in the art in light of this disclosure would understand how to administer a sufficient amount of the viral vector to a patient without undue experimentation.

Disclosed are pharmaceutical and other compositions containing the disclosed vectors. Such pharmaceutical and other compositions can be formulated so as to be used in any administration procedure known in the art. Such pharmaceutical compositions can be via a parenteral route (intradermal, intramuscular, subcutaneous, intravenous, or others). The administration can also be via a mucosal route, e.g., oral, nasal, genital, etc.

The disclosed pharmaceutical compositions can be prepared in accordance with standard techniques well known to those skilled in the pharmaceutical arts. Such compositions can be administered in dosages and by techniques well known to those skilled in the medical arts taking into consideration such factors as the breed or species, age, sex, weight, and condition of the particular patient, and the route of administration. The compositions can be administered alone, or can be co-administered or sequentially administered with other vectors or with other immunological, antigenic or therapeutic compositions. Such other compositions can include purified native antigens or epitopes or antigens or epitopes from the expression by a recombinant adenoviral or another vector system; and are administered taking into account the aforementioned factors.

Examples of compositions of the invention include liquid preparations for orifice, e.g., oral, nasal, anal, genital, e.g., vaginal, etc., administration such as suspensions, syrups or elixirs; and, preparations for parenteral, subcutaneous, intradermal, intramuscular or intravenous administration (e.g., injectable administration) such as sterile suspensions or emulsions. In such compositions the composition may be in admixture with a suitable carrier, diluent, or excipient such as sterile water, physiological saline, glucose or the like.

### V—Vaccines

Antigenic, immunological or vector compositions typically can contain an adjuvant and an amount of the vector or expression product to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryl lipid A, phospholipid conjugates such as those described by Goodman-Snitkoff et al. J. Immunol. 147:410-415 (1991), encapsulation of the protein within a proteoliposome as described by Miller et al., J. Exp. Med. 176:1739-1744 (1992), and encapsulation of the protein in lipid vesicles such as Novasome lipid vesicles (Micro Vescular Systems, Inc., Nashua, N.H.) can also be used.

The composition may be packaged in a single dosage form for immunization by parenteral (i.e., intramuscular, intradermal or subcutaneous) administration or orifice administration, e.g., perlingual (e.g., oral), intragastric, mucosal including intraoral, intraanal, intravaginal, and the like administration. And again, the effective dosage and route of administration are determined by the nature of the composition and by known factors, such as breed or species, age, sex, weight, condition and nature of host, as well as LD₅₀ and other screening procedures which are known and do not require undue experimentation. Dosages of expressed product can range from a few to a few hundred micrograms, e.g., 5 to 500 μg. A vaccine can be administered in any suitable amount to achieve an immune response at these dosage levels.

The carrier may also be a polymeric delayed release system. Synthetic polymers are particularly useful in the formulation of a composition having controlled release. An early example of this was the polymerization of methyl methacrylate into spheres having diameters less than one micron to form so-called nanoparticles, reported by Kreuter, J., Microcapsules and Nanoparticles in Medicine and Pharmacology, M. Donbrow (Ed). CRC Press, p. 125-148.

Microencapsulation has been applied to the injection of microencapsulated pharmaceuticals to give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the antigens are all factors that must be considered. Examples of useful polymers are polycarbonates, polyesters, polyurethanes, polyorthoesters and polyamides, particularly those that are biodegradable.

A frequent choice of a carrier for pharmaceuticals and more recently for antigens is poly (d,1-lactide-co-glycolide) (PLGA). This is a biodegradable polyester that has a long history of medical use in erodible sutures, bone plates and other temporary prostheses where it has not exhibited any toxicity. A wide variety of pharmaceuticals including peptides and antigens have been formulated into PLGA microcapsules. A body of data has accumulated on the adaption of PLGA for the controlled release of antigen, for example, as reviewed by Eldridge, J. H., et al. Current Topics in Microbiology and Immunology. 1989, 146:59-66. The entrapment of antigens in PLGA microspheres of 1 to 10 microns in diameter has been shown to have a remarkable adjuvant effect when administered orally. The PLGA microencapsulation process uses a phase separation of a water-in-oil emulsion. The compound of interest is prepared as an aqueous solution and the PLGA is dissolved in a suitable organic solvents such as methylene chloride and ethyl acetate. These two immiscible solutions are co-emulsified by high-speed stirring. A nonsolvent for the polymer is then added, causing precipitation of the polymer around the aqueous droplets to form embryonic microcapsules. The microcapsules are collected, and stabilized with one of an assortment of agents (polyvinyl alcohol (PVA), gelatin, alginates, polyvinylpyrrolidone (PVP), methyl cellulose) and the solvent removed by either drying in vacuo or solvent extraction.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE TM or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18^{th} edition, Mack Publishing Company, (ed.) 1980).

Adjuvants may also be included. Adjuvants include, but are not limited to, mineral salts (e.g., AIK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC31; see Schellack, C. et al (2003) Proceedings of the 34th Annual Meeting of the German Society of Immunology; Lingnau, K. et al (2002) Vaccine 20(29-30): 3498-508), JuvaVax® (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3DMPL), imiquimod (also known in the art as IQM and commercially available as Aldara®; U.S. Pat. Nos. 4,689,338; 5,238,944; Zuber, A. K. et al (2004) 22(13-14): 1791-8), and the CCR5 inhibitor CMPD167 (see Veazey, R. S. et al (2003) J. Exp. Med. 198: 1551-1562). Aluminum hydroxide or phosphates (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, in the same viral vectors as those encoding the antigens of the invention or on separate expression vectors. Alternatively, vaccines of the invention may be provided and administered without any adjuvants.

The immunogenic compositions can be designed to introduce viral proteins to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the vectors in the immunogenic compositions can be readily determined by those of skill in the art. For example, the dosage of the virus can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. In a particularly advantageous embodiment of the present invention, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks.

The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

### EXAMPLES

The following examples are illustrative of disclosed methods. In light of this disclosure, those of skill in the art will recognize that variations of these examples and other examples of the disclosed method would be possible without undue experimentation.

### Example 1

### PBMC T Cell Assays

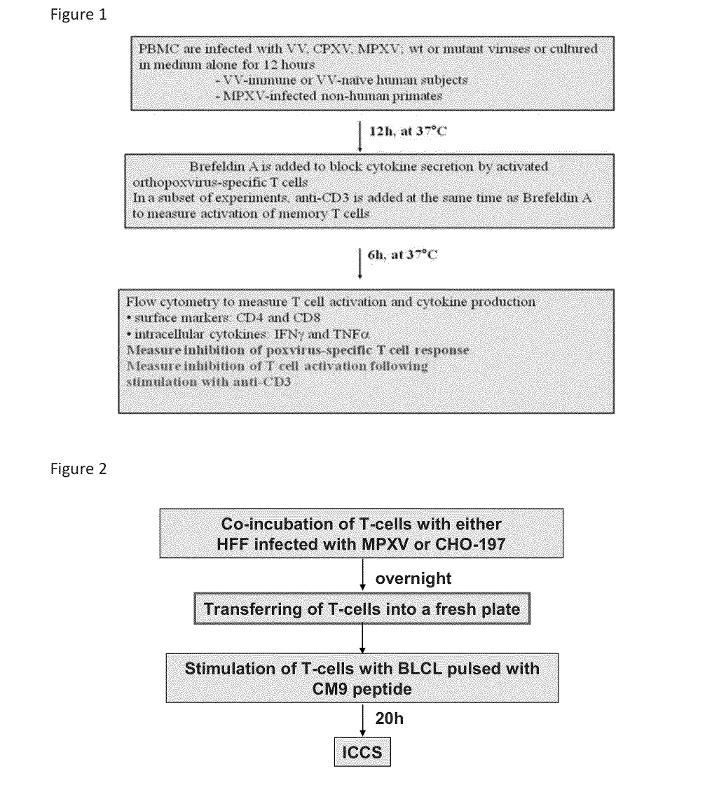

Referring now to FIG. 1, for MHC-dependent stimulation, PBMC were cultured at 37° C. with 6% CO₂ in RPMI containing 20 mM HEPES, L-glutamine, antibiotics, and 5% FBS, with or without the indicated virus at an MOI of 0.3 for 12 hours. Viruses were purified by sucrose gradient. Brefeldin A (ICN Biomedicals Inc., Costa Mesa, Calif.) was added at a final concentration of 2 μg/mL for an additional 6 hours.

For MHC-independent stimulation, PBMC were co-incubated with αCD3 Ab and 2 μg/mL Brefeldin A (ICN Biomedicals Inc., Costa Mesa, Calif.) for 6 hours. The cells were stained overnight at 4° C. with antibodies specific for CD8β (clone 2ST8.5H7, Beckman Coulter) and CD4 (clone L200, BD Biosciences PharMingen, San Diego, Calif.). Cells were fixed, permeabilized and stained intracellularly using antibodies to IFNγ (clone 4S.B3, eBioscience Inc., San Diego, Calif.) and TNFα (clone Mab11, eBioscience). Samples were acquired on an LSRII (Beckton Dickinson), acquiring approximately 1-2 million events per sample. Data was analyzed using FloJo software (Tree Star). Non-viable cells were excluded using a live cell gate based on the viability stain, Aqua (LIVE/DEAD® Fixable Dead Cell Stain, Invitrogen), followed by an optimized lymphocyte gate based on forward and side scatter characteristics. The number of virus-specific IFNγ⁺TNFα⁺ T cells was determined after gating on live CD4⁻/CD8β⁺ T cells and subtracting the number of IFNγ⁺TNFα⁺ events from uninfected cultures.

FIG. 2 describes a monkey CM9-specific T-cell assay. This assay is based on MHC-dependent stimulation of CD8+ T cell lines obtained from SIV-infected rhesus macaques (RM) that are specific for the SIVgag181-189 peptide (CTPYDINQM) (CM9) presented by the RM-specific MHC molecule MamuA01. T-cells were co-incubated with cells expressing SEQ ID NO: 1-either MPXV infected HFF-cells or CHO-197, a stably transduced cell line engineered to express SEQ ID NO: 1. After about 18 hours of co-incubation, T-cells were washed and transferred into a fresh plate for stimulation with BLCL cells pulsed with CM9 peptide in the presence of BFA. The percentage of INFγ⁺ TNFα⁺ T-cells was determined by ICCS as described above

### Example 2

### Determination of the MPXV ORF Responsible for Evasion of T-Cell Stimulation

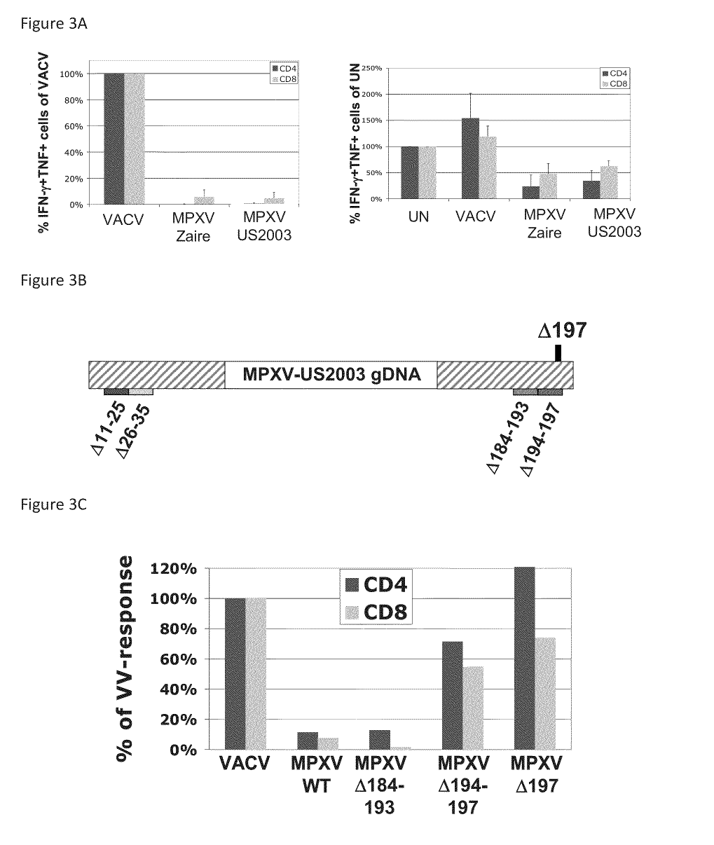

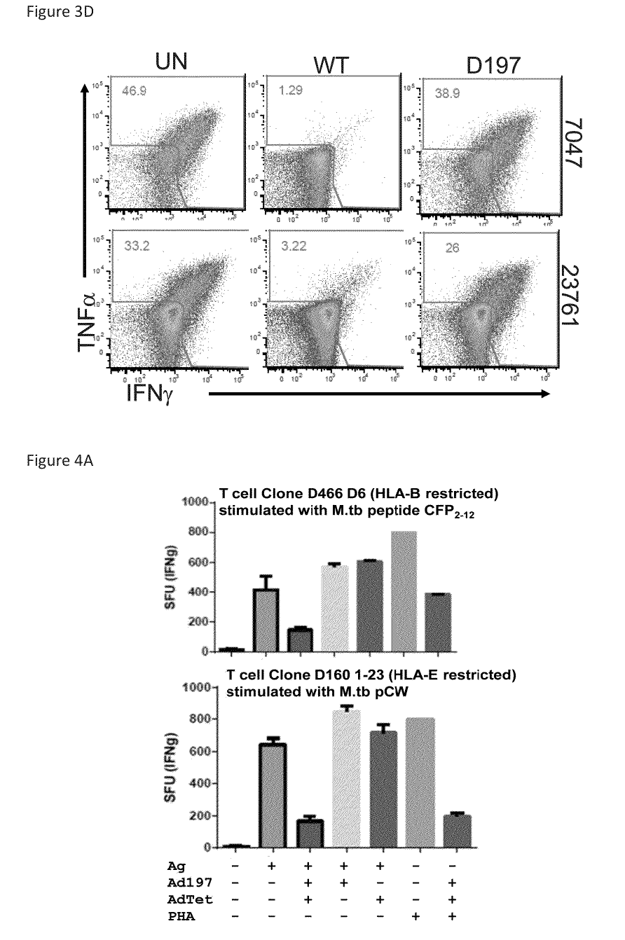

FIG. 3A shows that the MPXV US 2003 strain inhibits both MHC-dependent (left) and -independent (right) human PBMC T-cell stimulation. The responses of CD4⁺ (black) and CD8⁺ (grey) T-cells were tested using PBMC T-cell assay described in FIG. 1. Vaccinia virus was used as a negative control since it does not inhibit T-cell stimulation. The MPXV Zaire strain used as a positive control was shown to efficiently block both MHC-dependent and independent stimulation.

FIG. 3B shows the location of 10 Kb deletions (colored boxes) or a single ORF 197 deletion (black box) were created in the terminal regions of MPXV gDNA (hashed boxes) non-essential for viral replication using in-vivo homologous recombination. The deleted regions were replaced with a cassette expressing green fluorescent protein (GFP) and guanine-hypoxanthine phosphoribosyltransferase (GPT) selection markers. To verify the deletion and the absence of contaminating wild-type virus gDNA of the mutant virus was purified with DNeasy kit (QIAGEN, Valencia, Calif.) and tested by PCR using and primers specific to the deleted region and its flanking regions.

FIG. 3C shows MPXV US 2003 deletion mutants tested by human PBMC T-cell assay. CD4 (black) and CD8 (grey) T-cell responses to wild-type MPXV US2003 and the mutants, Δ184-193, Δ194-197, Δ197 were tested using MHC-dependent stimulation of human PBMC as described FIG. 1. The MPXV Δ197 mutant resulted in worse blocking of MHC dependent CD4 T cells stimulation than vaccinia virus and blocking of CD8 T cells almost 80% that of vaccinia virus.

FIG. 3D shows the MPXV US 2003 Δ197 mutant tested using the monkey CM9-specific CD8+ T-cell assay described in FIG. 2. HFF cells were infected with indicated viruses at an MOI of 2. Infection rates for the wild-type and Δ197 mutant viruses were 47% and 49%, respectively. At 4hpi, HHF cells were washed and overlaid with monkey CM9-specific CD8+ T-cells as described in FIG. 2. After 18 hours of co-incubation, T-cells were washed and transferred into a fresh plate for stimulation with CM9-peptide pulsed BLCL cells. The percentage of INFγ⁺ TNFα⁺ T-cells was determined by ICCS. Cells infected with the Δ197 mutant virus were more efficiently activated in two individuals.

### Example 3

### Use of SEQ ID NO: 1 to Inhibit T Cell Activation In Vitro

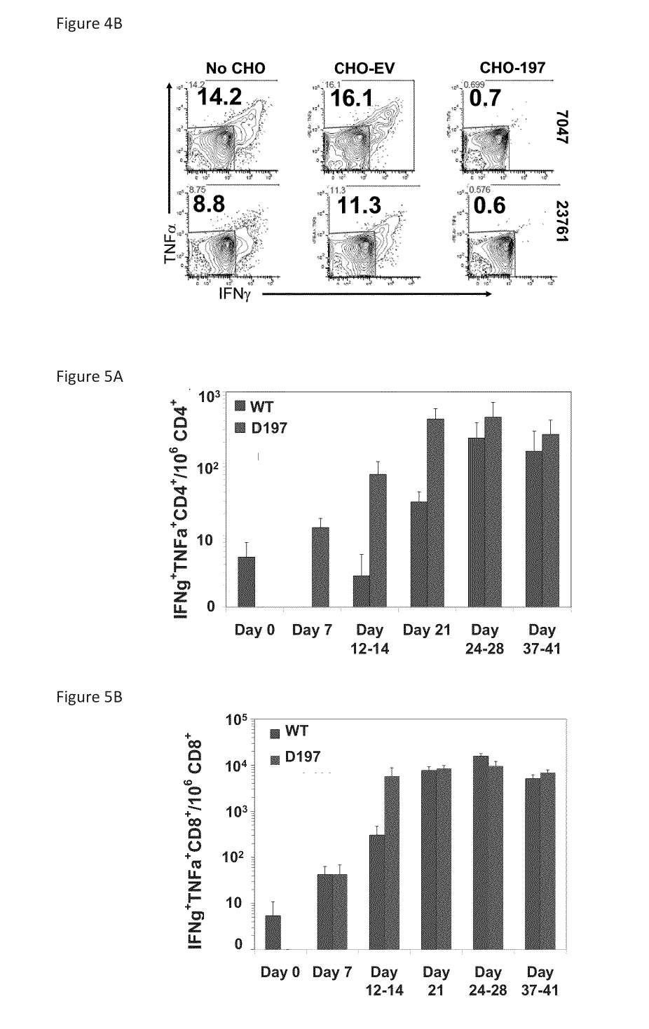

FIG. 4 shows that SEQ ID NO: 1 is capable of inhibiting T cell activation in response to a wide variety of stimulation vitro. In FIG. 4A, SEQ ID NO: 1 expressed in an adenoviral vector inhibits human Mtb-specific T cells. Mtb-specific HLA-B (top) and HLA-E (bottom) restricted CD8⁺ T cell clones were incubated for 18 hours with BEAS-2B cells transduced (for 72 hrs) with an adenovirus comprising SEQ ID NO: 1 or an empty control in the presence of antigen or PHA. IFN-γ production by T cells was detected by ELISPOT.

In FIG. 4B, SEQ ID NO: 1 expressed by a stably transfected CHO cell line inhibits monkey CM9-specific CD8 T-cell responses. CHO-EV, a negative control cell line transduced with an empty vector or CHO cells expressing SEQ ID NO: 1 were co-incubated with monkey CM9-specific CD8+ T-cells overnight. After this, T-cells were washed and transferred into a fresh plate for stimulation with CM9-peptide pulsed BLCL cells as described in FIG. 2. The percentage of INFγ⁺ TNFα⁺ T-cells was determined by intracellular cytokine staining.

### Example 4

### In Vivo Attenuation of MPXVΔ197

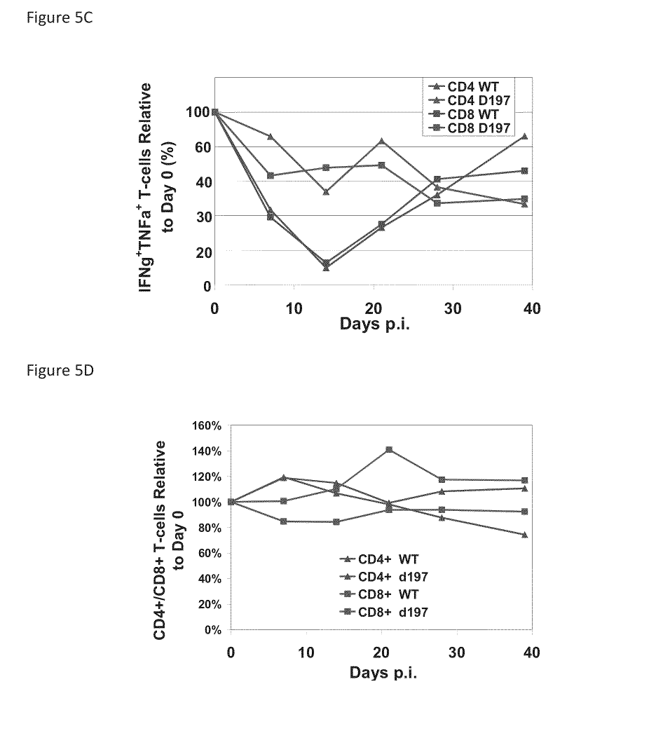

In FIG. 5, 8 rhesus macaques were inoculated intrabronchially with 2×10⁵ PFU MPXVUS2003 or MPXVΔ197 at day 0 post infection (p.i.). PBMC were purified from whole blood on the indicated days post infection. FIG. 5A depicts the PBMC CD4⁺ responses to MHC dependent stimulation for wild type (blue) or MPXΔ197 (red) animals. FIG. 5B depicts the CD8⁺ responses. The assay was performed as described in FIG. 1. PBMC were stimulated with vaccinia virus (0.3 MOI). The graph shows the number of IFNγ⁺ TNFα⁺ T-cells as determined by intracellular cytokine staining relative the number of days post infection.

FIG. 5C depicts the PBMC CD4⁺ T-cell responses to MHC-independent stimulation for WT (blue) or MPXVΔ197 (red) infected animals. FIG. 5D depicts the same for PBMC CD8⁺ T cells. PBMC were stimulated with αCD3 and assayed by intracellular cytokine staining for IFNγ and TNFα expression as described in FIG. 1. The graph shows the percentage of the responsive T cells relative to day 0 post infection. FIG. 5D depicts Average percent of CD4+ and CD8+ T-cells in PBMC for WT (blue) and MPXV Δ197 (red) infected animals by ICCS.

### Example 5

### MPXV197 is Essential for T Cell Inactivation by Monkeypoxvirus

MPXV Zaire is a strain of monkeypox virus known to inhibit CD4+ and CD8+ T-cell activation by both MHC-dependent and MHC-independent stimuli. MPXV Zaire encodes a homolog of CPXV203, which was previously known to cause T cell evasion in cowpox. The monkeypoxvirus strain MPXV US2003 is known to lack most of the CPXV203 homolog (Likos A M et al, J Gen Virol 86, 2661-2672, (2003); incorporated by reference herein). Because vaccinia virus-specific T cells recognize cells infected with UV-inactivated monkeypoxvirus, human PBMC from donors recently immunized with vaccinia virus were infected in vitro with MPXV Zaire and US2003 at an MOI of 0.3. T cell responses were then analyzed by intracellular cytokine staining (ICCS) for TNFα and IFNγ. Cells were also infected with vaccinia virus as a control since it is known not to inhibit T cell responses.

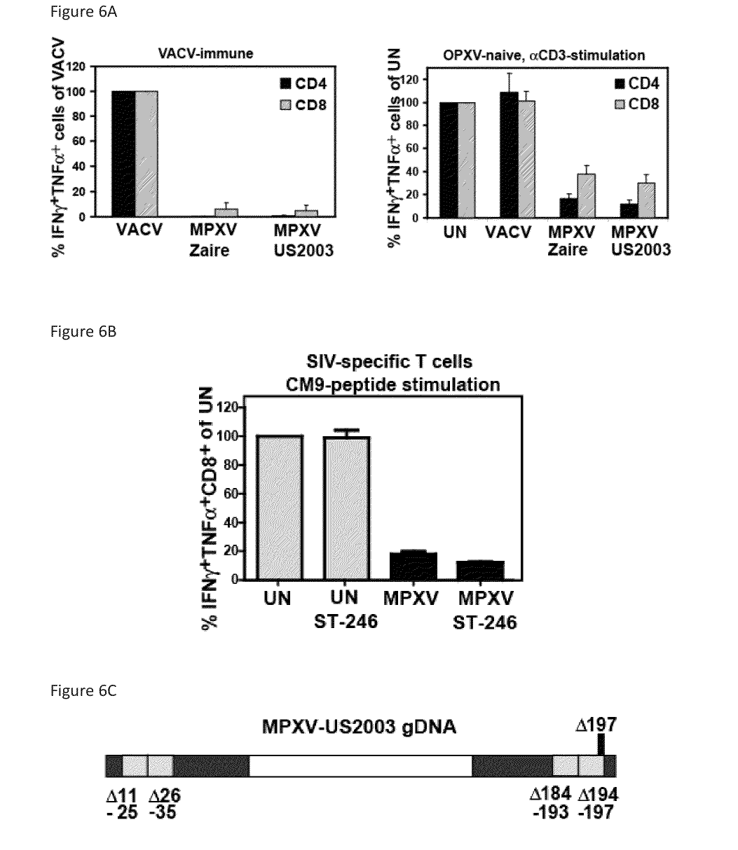

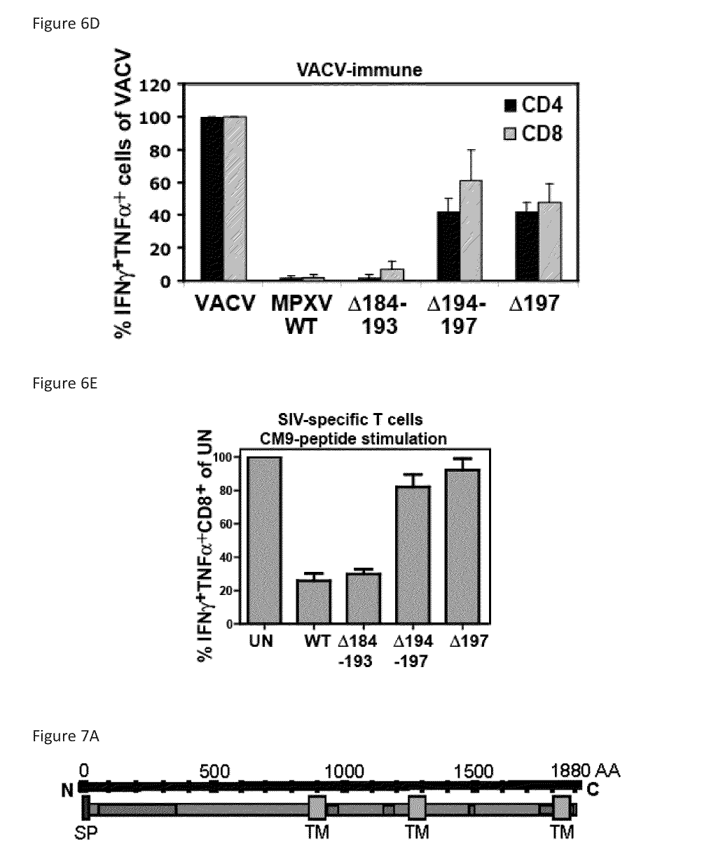

Vaccinia virus resulted in a vigorous virus-specific CD4⁺ and CD8⁺ T cell response. However, cells infected with either MPXV Zaire or MPXV US2003 had only 6% of the TNFα+, IFNγ+ cells of the control (FIG. 6A). Additionally, T cell activation by plate-bound αCD3 Ab in the presence of MPXV Zaire and US2003 was examined. As shown in FIG. 6A (right panel), MPXV US2003 also is capable of inhibiting MHC-independent T cell stimulation. The data therefore show that the monkeypoxvirus homolog of CPXV203 is not required for inhibition of T cells.

### Example 6

### Monkeypox Virus Inhibits T Cell Activation not by Infecting T Cells, but by Acting in Trans

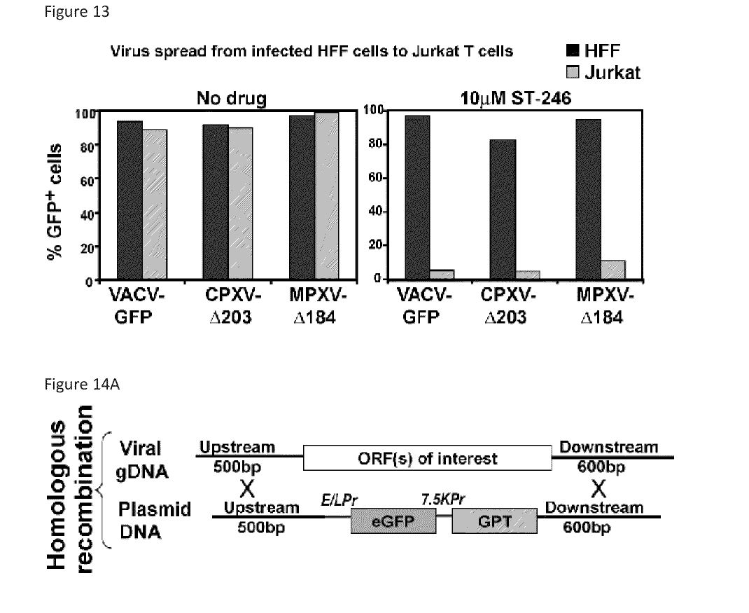

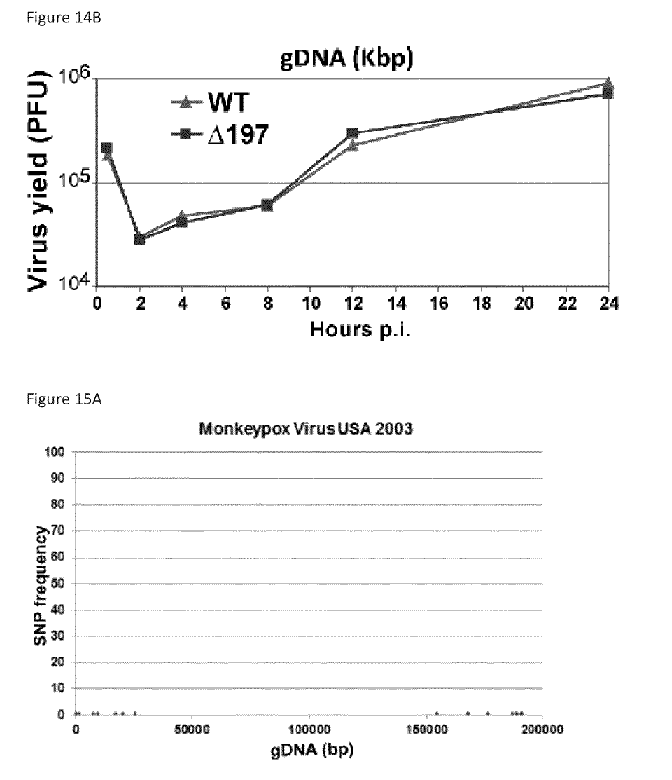

In PBMC, OPXV infect CD14⁺ monocytes but rarely infect T cells. However, to rule out with certainty that MPXV inhibits T cells directly, an experiment that separated MPXV-infection from antigen presentation was performed. Human foreskin fibroblasts (HFF) were infected with MPXV and co-incubated with rhesus macaque (RM)-derived T cell lines specific for the MaMu-A*01-restricted SIV GAG₁₈₁₋₁₈₉epitope CM9 (Loffredo J T et al, J Virol 81, 2624-2634 (2007); incorporated by reference herein. Autologous B cells immortalized by simian lymphocryptovirus (BLCLs) were used as antigen presenting cells. So in this assay the infected cells (the HFF) do not contribute to T cell stimulation. Instead, that is provided by peptide-pulsed BLCLs. Compound ST-246 was used to inhibit egress of viral particles from infected cells (Yang G et al, J Virol 79, 13139-13149 (2005); incorporated by reference herein). Control experiments demonstrated that ST246 efficiently (˜90%) prevented spread of vaccinia virus, cowpoxvirus, and monkeypoxvirus to Jurkat T cells (FIG. 13).

When ST-246-pretreated HFF were infected with MPXV, T cell stimulation by CM9 peptide-pulsed BLCLs was <10% of the uninfected cell control (FIG. 6B) confirming that MPXV inhibits T cell activation by not acting directly upon T cells. Since the T cell inhibitory factor is not secreted, this process most likely involves cell to cell contact.

### Example 7

### Identification of MPXV197 as the Gene Required for T Cell Evasion in Monkeypox

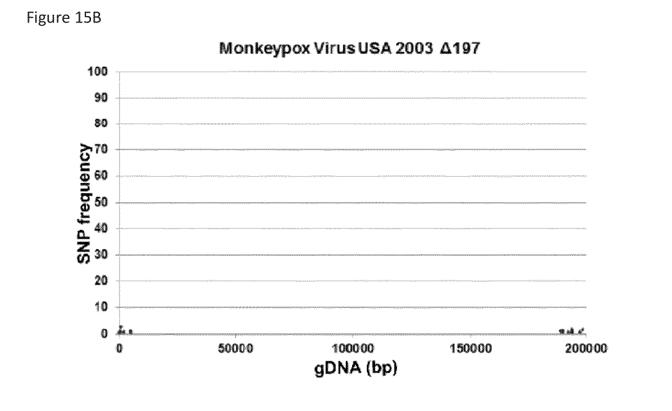

Four deletion mutants, each lacking about 10 kb of the termini of the MPXV US2003 genome were generated (FIG. 6C FIG. 14A). Each mutant was examined for its ability to inhibit stimulation of T cells in PBMC from vaccinia immune human subjects (FIG. 6D) or peptide-stimulation of CM9-specific T cells from RM (FIG. 6E). Mutants lacking ORFs 11-25, 26-35, or 184-193 did not activate poxvirus-specific T cells (FIG. 6E), but still inhibited peptide-stimulation of CM9-specific T cells (FIG. 6E). Monkeypoxvirus lacking ORFs 194-197 activated both CD4+ and CD8+ T cells in VACV-immune PBMC (FIG. 6D) and no longer inhibited peptide stimulation of CM9-specific T cells (FIG. 6E), indicating that the MPXV194-197 region encodes the T cell inhibitor. A second mutation with a deletion of only MPX197 (MPXΔ197) was made. As shown in FIG. 6D and FIG. 6E, MPXVΔ197 stimulated poxvirus-specific CD4+ and CD8+ T cells similar to VACV and peptide stimulation of CM9-specific T cells was no longer inhibited.

### Example 8

### Cellular Localization of MPX197

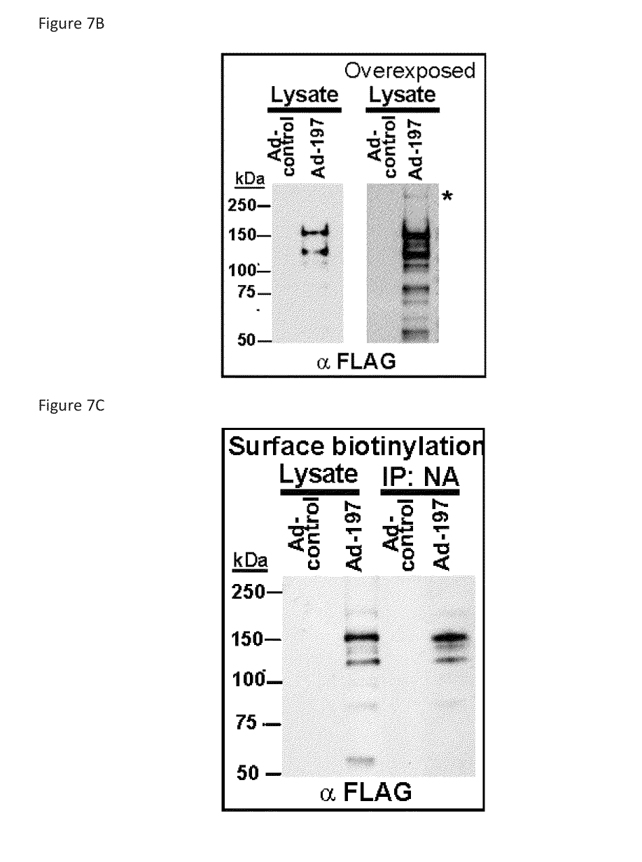

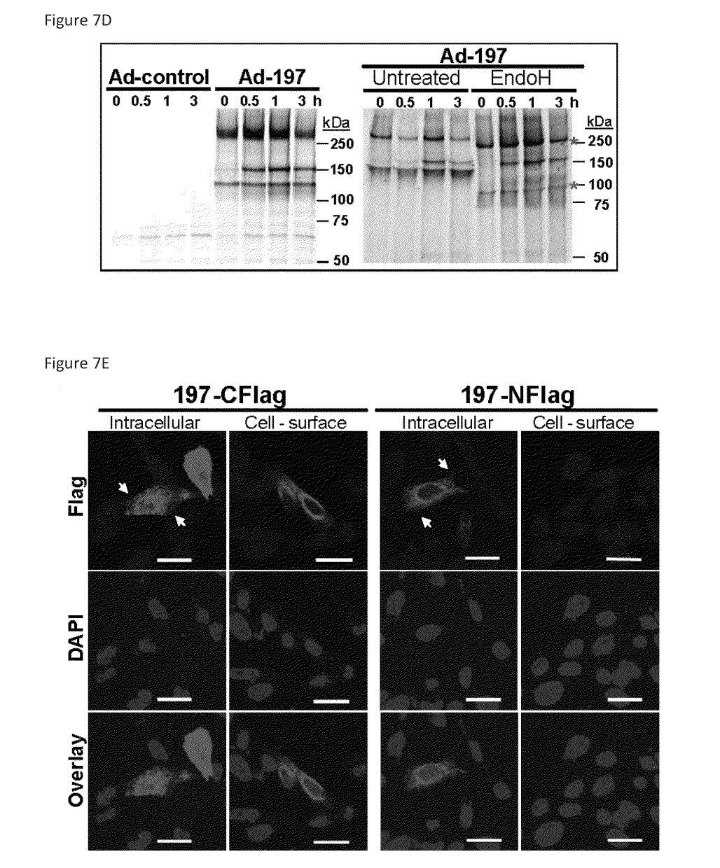

Wild type MPX197 was cloned into a plasmid expression vector, but the gene was not expressed. Sequence analysis of both the wild type MPX197 and VARV B22R indicated a number of RNA splicing signals and other sequences that destabilize RNA. These were removed through the generation of codon optimized sequences that were cloned into both plasmid and adenovirus expression vectors, resulting in RNA and protein expression. MPXV197 is the largest ORF in the genome of MPXV encoding for 1880 amino-acids with a predicted molecular mass of 212 kDa, a predicted cleavable N-terminal signal peptide (SP), multiple N-glycosylation sites, a C-terminal transmembrane (TM) domain, and potentially one or more internal TM domains (FIG. 7A). Transient expression of MPXV197 in CHO cells and immunoblotting with αFLAG-Ab revealed two predominant bands with apparent molecular mass of ˜150 kDa, and ˜140 kDa and several minor, smaller bands as well as a large protein >250 kDa (FIG. 7B). Surface biotinylation followed by streptavidin-precipitation and immunoblot with αFLAG antibody was performed. The ˜150 kDa species was the predominant species recovered (FIG. 7C). Pulse-chase labeling revealed that the ˜150 kDa protein was a processing product derived from the large >250 kDa precursor protein. The ˜140 kDa fragment carrying the C-terminal Flag-tag was synthesized simultaneously with the large precursor protein suggesting that this fragment is derived from an internal start site (FIG. 7D). Endoglycosidase H (EndoH)-treatment reduced the apparent molecular mass of the largest and the smaller fragment whereas the ˜150 kDa processing product was unaffected by EndoH treatment. Taken together, these results suggest that the full-length 250 kDa protein is processed into a ˜150 kDa fragment that is transported beyond the ER to the cell surface. The C-terminal location of the FLAG-tag identifies the ˜150 kDa fragment as a C-terminal fragment.

Further sub-cellular localization of MPXV197 was determined by immunofluorescence analysis (IFA) using confocal laser scanning microscopy (CLSM). Staining with αFLAG Ab of permeabilized or non-permeabilized CHO cells revealed that the C-terminus of MPXV197 locates to the extracellular face of the plasma membrane (FIG. 7E). In contrast, N-terminally Flag-tagged MPXV reacted with αFLAG Ab only when cells were permeabilized (FIG. 7E) consistent with the full-length protein and potential N-terminal fragments remaining intracellular. The extracellular location of the C-terminus suggests that the C-terminal fragment most likely displays a multi-transmembrane topology with additional parts being exposed extracellularly (FIG. 7A).

### Example 9

### Expression of MPX197 Confers Inhibition of T Cell Stimulation on CHO Cells

Adenovirus expressing MPX197 under the control of a tetracycline regulated promoter (Ad-197) was used to transfect CHO cells. The CHO cells were then transduced with CM9-specific CD8+ T cells stimulated with peptide-pulsed BLCL. So in this assay, T cells were stimulated by exposure to cognate peptides presented by BLCLs but MPXV197 was provided indirectly by expression in the non APC CHO cells. Adenovirus expressing the tetracycline transactivator (Ad-tTA) was cotransfected with Ad-197 to activate expression of MPX197.

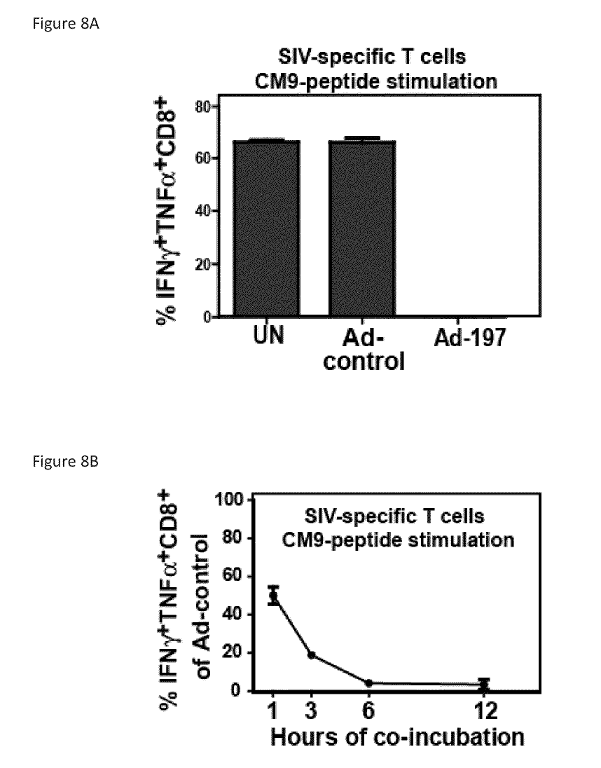

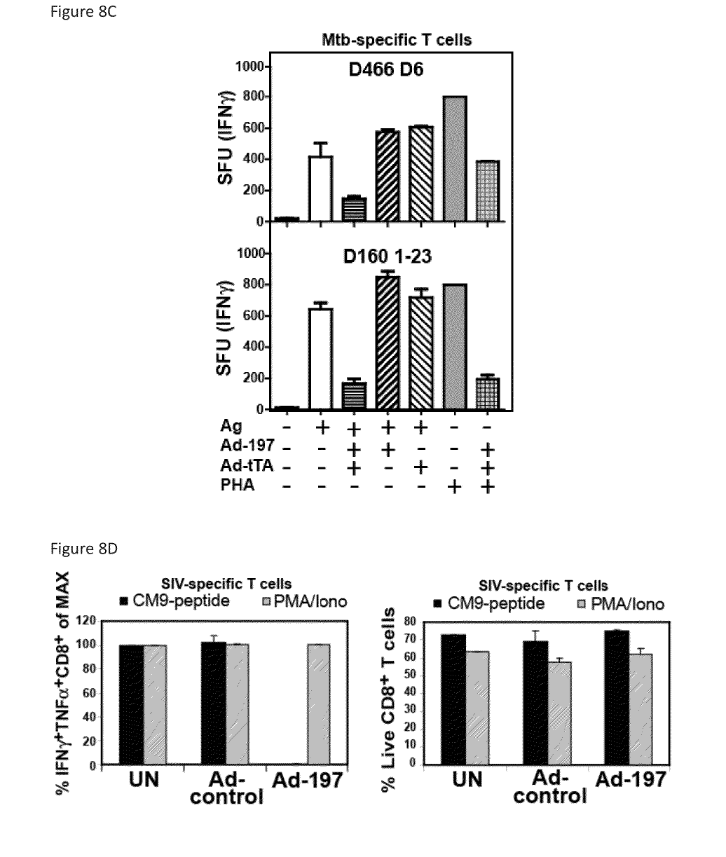

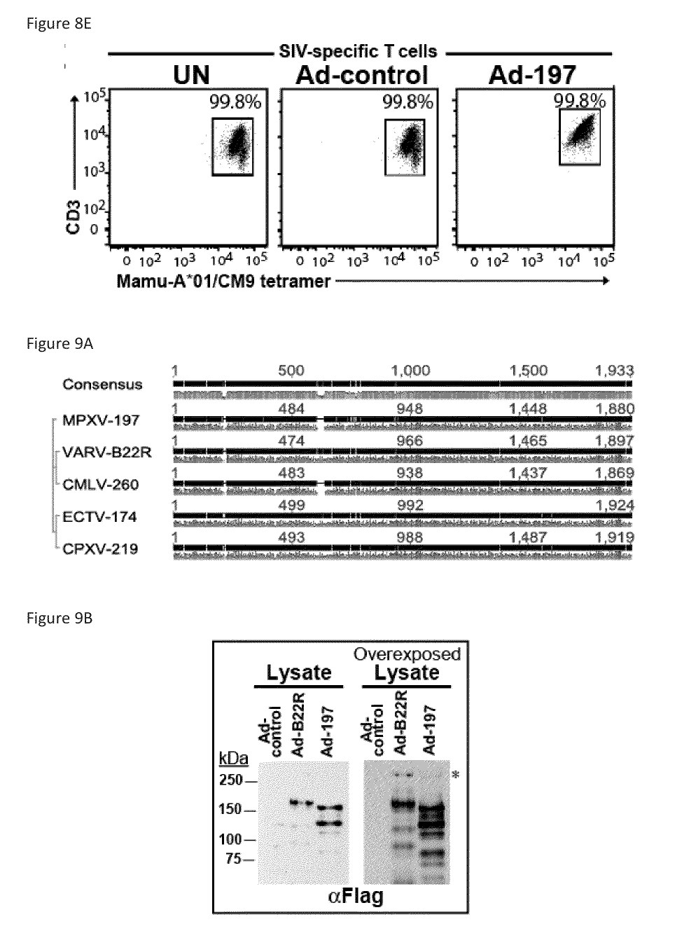

CHO cells transfected with Ad-tTA alone did not inhibit T cell activation with cognate antigen (Ad-control, FIG. 8A). In contrast, T cell responses were reduced to ˜0.01% of control upon cotransfection of Ad-tTA and Ad-197 resulting in MPXV197 expression (Ad-197, FIG. 8A). Thus, MPXV197 inhibits T cell stimulation even when provided by unrelated cells of a different species. To measure the kinetics of the CM9-specific CD8+ T cell inactivation, CM9-specific T cells were co-incubated with MPXV197-expressing cells for variable time periods prior to stimulation with peptide pulsed BLCLs. Inhibition of T cell stimulation was observed at 1 hour of exposure to MPXV197, with maximal inhibition at 6 hours (FIG. 8B).

### Example 10

### MPXV197 Inhibits Antigen Independent and Antigen Dependent CD8+ T Cell Stimulation

The ability of MPXV197 to inhibit M. tuberculosis-specific CD8+ T cell clones D466 D6 recognizing peptide CFP2-12 presented by HLA-B (Lewinsohn D A et al, PLoS Pathog 3, 1240-1249 (2007); incorporated by reference herein) and D160 1-23 which is stimulated by pronase digested Mtb cell wall in the context of HLA-E (Heinzel A S et al, J Exp Med 196, 1473-1481 (2002); incorporated by reference herein) was determined. BEAS-2B epithelial cells were infected with either Ad-197 alone or together with Ad-tTA followed by incubation with Mtb-specific CD8+ T cell clones. As shown in FIG. 8C, stimulation of both clones was inhibited by the expression of MPXV197.

This assay differs from the previous assay in that MPXV197 is expressed in the antigen presenting cells, so MHC- and antigen-independent T cell stimulation of these T cell clones was assessed. T cells were treated with phytohaemagglutinin (PHA), a lectin that activates the TCR non-specifically by carbohydrate cross-linking. PHA stimulation of both D466 D6 and D160 1-23 was inhibited by MPXV197. This indicates that MPXV197 inhibits T cell stimulation regardless of the type of TCR stimulus.

### Example 11

### MPXV196 Suppression of T Cell Stimulation is Upstream of PKC and does not Result in Cell Death

A lack of cellular amine-reactive fluorescent staining (LIVE/DEAD Fixable Dead Cell Stain) indicates that T cell membranes remain intact in the presence of MPXV197 (FIG. 8D, right panel). Additionally, CM9-specific T cells were stimulated with phorbol 12-myristate 13-acetate (PMA) which activates protein kinase C (PKC) and the Ca²⁺ ionophore ionomycin (lono). Unlike peptide stimulation, MPXV197-expressing CHO cells did not inhibit T cell stimulation by PMA/lono (FIG. 8D, left panel). Thus, T cells remain viable after exposure to MPXV197 suggesting that MPXV197 counteracts TCR-dependent signal transduction upstream of PKC. Moreover, exposure of CM9-specific CD8+ T cells to MPXV197-expressing CHO cells did not impair their ability to bind a Matsu-A*01/CM9 tetramer suggesting that MPXV197 does not interfere with MHC-I peptide loading (FIG. 8E).

### Example 12

### Inactivation of T Cell Stimulation by MPXV197 Homologs

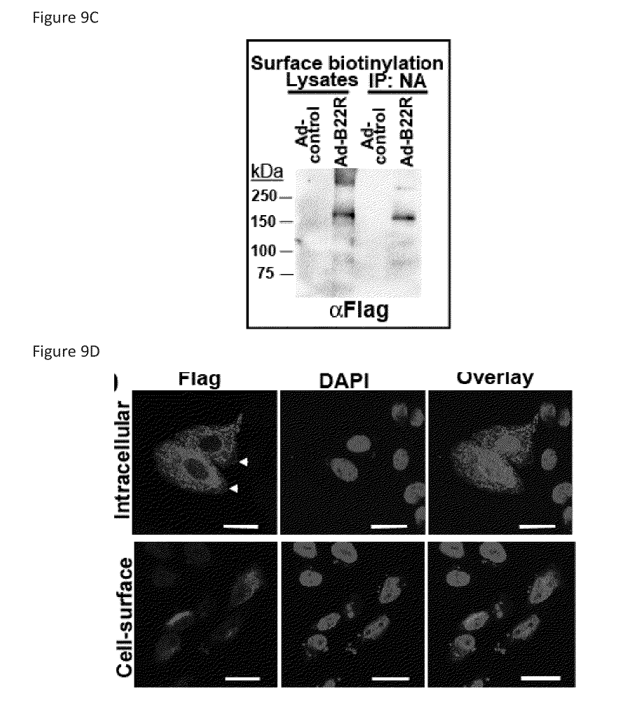

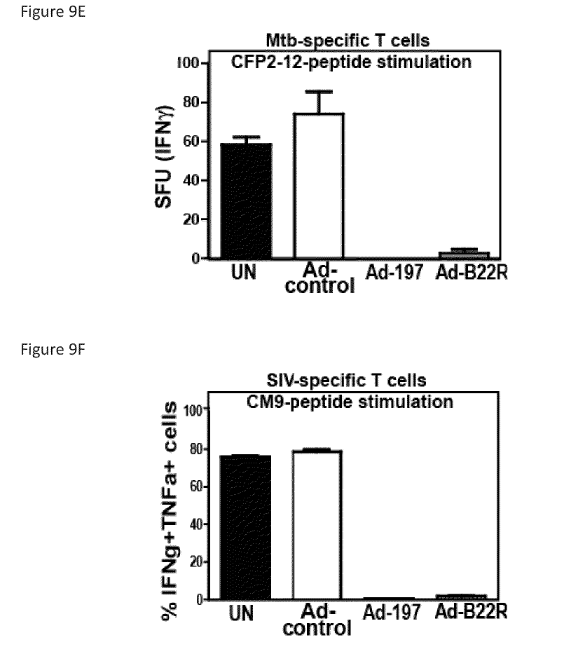

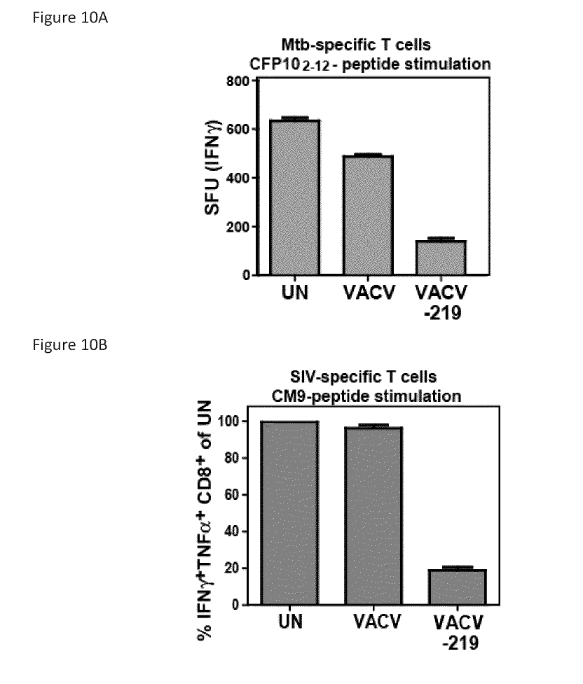

MPXV197 belongs to the B22-protein family found in some but not all orthopoxviruses (FIG. 9A). These include CPXV219 from cowpoxvirus with 84% amino-acid identity and B22 from variola virus with 86% amino-acid identity. A codon-optimized 1897aa variola virus B22 (VARV B22) with a predicted molecular mass of ˜214 kDa was cloned into expression vectors. Similar to MPXV197, immunoblots and surface biotinylation of VARV B22 revealed a surface expressed ˜150 kDa fragment with the B22 fragment being slightly larger than the corresponding MPXV197 fragment (FIG. 9B and FIG. 9C). Also similar to MPXV197 was that the full-length precursor protein was barely detectable with the 150 kDa protein being the final product. The smaller protein bands were less abundant than those seen in MPXV197-expressing cells. Additionally, the C-terminus of VARV B22 is exposed at the cell surface (FIG. 9D). T cell inhibition by VARV B22 was examined using both human Mtb-specific CD8+ T cell clones and rhesus CM9-specific CD8+ T cell lines as described above. As shown in FIG. 9E and FIG. 9F, VARV B22 inhibited T cell stimulation of both human and RM T cells as efficiently as MPXV197.

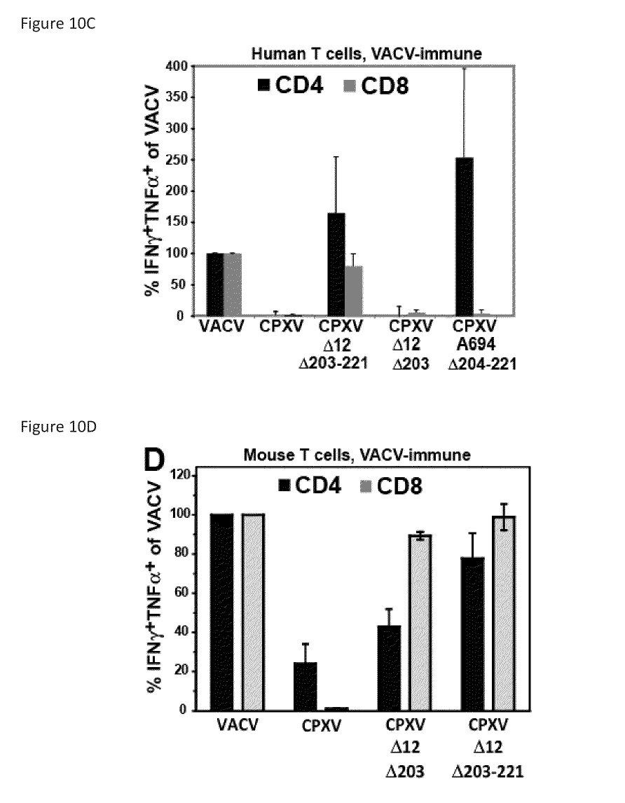

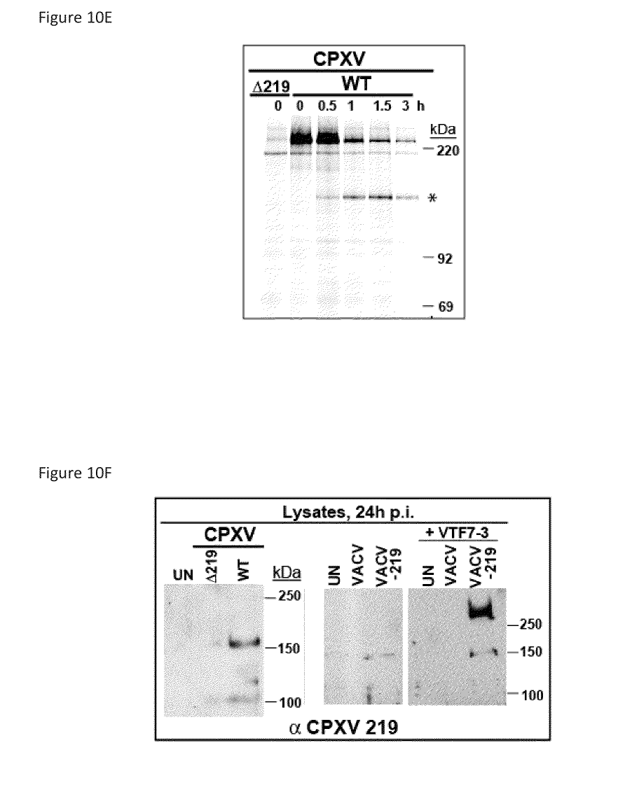

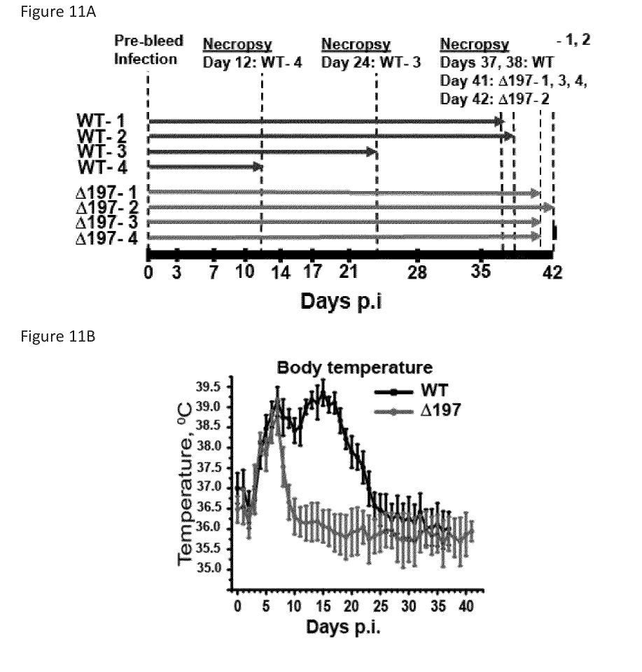

A recombinant vaccinia virus expressing CPXV219 (VACV-219) was used to infect BEAS-2B cells and to test whether its expression inhibited the stimulation of Mtb-specific T cells or to infect HFF and monitor stimulation of SIV-specific T cells by CM9 peptide loaded BCBLs as described above. While vaccinia virus did not impact stimulation of human or RM T cells, VACV-219 inhibited T cell stimulation in both instances (FIG. 10A and FIG. 10B).

The finding that CPXV219 inhibits T cells was unexpected since it was previously reported that poxvirus-specific T cells were stimulated once MHC-I-dependent antigen presentation by CPXV was restored due to deletion of CPXV12 and CPXV203. However, genome analysis of our deletion virus CPXVΔ12Δ203 revealed that, upon passaging, this mutant had acquired additional deletions downstream of CPXV203 due to a recombination event resulting in ORF204-221 being replaced by a duplication of ORF10-11. Therefore, this deletion virus (now designated CPXVΔ12Δ203-221) lacks not only CPXV12 and CPXV203, but also CPXV219.

An independently generated CPXVΔ12Δ203 mutant was also reported to stimulate poxvirus-specific T cells (Byun M et al 2009 supra), although this analysis was limited to murine T cells. As a result, cowpoxvirus with a deletion of CPXV219 alone, with a single deletion of either CPXV12 or CPXV203 or with a deletion of both CPXV12 and CPXV23. Stimulation of poxvirus-specific human T cells was analyzed by infecting PBMC from VACV-immune subjects with the cowpoxvirus subsets and monitoring T cell activation by ICCS. Stimulation of murine T cells was monitored by adding splenocytes from VACV-immunized mice to cowpoxvirus-infected A20 cells (FIG. 10C and FIG. 10D).

An unmutated cowpoxvirus did not stimulate poxvirus-specific human CD8+ and CD4+ T cells. However, the mutant termed A694 lacking the genomic region CPXV204-221 stimulated human CD4+ T cells but not CD8+ T cells (FIG. 10C). Since A694 contains CPXV12 and CPXV203 these data suggest that human CD8+ T cells are not stimulated due to MHC-I evasion whereas human CD4+ T cells were stimulated due to the absence of CPXV219. A mutant with deletions of all of CPXV12, CPXV203 and CPXV219 (CPXVΔ12Δ203-221) stimulated both human CD4+ and CD8+ T cells. A mutant termed CPXVΔ12Δ203 which has a deletion in both CPXV12 and CPXV203 but has an active CPXV 219 did not stimulate poxvirus specific human CD8+ T cells (FIG. 10C). These results are consistent with a model by which CPXV219 inhibits both human CD4+ and CD8+ T cells by a similar mechanism to that of MPXV197.

However, when stimulation of murine poxvirus-specific T cells was examined with the same series of mutants, CD8+ T cells were stimulated in the absence of CPXV12 and CPXV203 even when CPXV219 was present (FIG. 10D). The CPXVΔ12Δ203 mutant showed reduced activation of CD4+ T cells compared to VACV or CPXVΔ12Δ203-221. This result suggests that CPXV219 does not efficiently inactivate murine CD8+ T cells but might impact murine CD4+ T cells. These results indicate that B22 proteins inhibit human and monkey T cells, but are less active against murine T cells.

The expression of GST-tagged CPXV219 in cowpox infected human 143 cells and CHO cells as well as in HEK 293 cells infected with VACV-219 was assessed using a rabbit antiserum (Fi). CPXV219 was expressed with early kinetics and detectable as early as 3 hours post infection. (data not shown). Metabolic pulse/chase labeling and immunoprecipitation at 3 hours post infection further demonstrated that a high molecular mass product (>220 kDa) was processed into a ˜150 kDa fragment (FIG. 10E). A similarly sized protein was the predominant fragment in immunoblots of cowpox-infected CHO cells. This fragment was absent from CPXVΔ219-infected cell lysates (FIG. 10F). Similarly, a ˜150 kDa fragment was the predominant protein found in VACV-219 infected cells in the absence of the T7 polymerase. However, upon co-infection with T7-polymerase expressing VACV, the >250 kDa precursor was highly expressed whereas the ˜150 kDa fragment was only slightly increased consistent with the majority of the protein remaining in the ER-resident precursor state upon overexpression.

These data suggest that in both virally infected and ectopically expressing cells the full-length precursor protein is processed into a ˜150 kDa fragment. Since the anti-CPXV219 antiserum was raised against the whole protein, it is not known which part of the protein is recognized. However, the ˜150 kDa fragment of both MPXV197 and VARV B22 was detected by a C-terminal FLAG-tag suggesting that the CPXV219 ˜150 kDa fragment is likewise C-terminal. Therefore, a ˜150 kDa C-terminal fragment is the ultimate product of MPXV197, VARV B22 and CPXV219 and that this fragment is transported to the cell surface where it acts as T cell inactivator.

### Example 13

### MPXVΔ197 can be Used to Generate an Immune Response In Vivo

Since B22 proteins are more active against primate than rodent T cells, a recently described intrabronchial (i.b.) inoculation model in RM Estep R D et al, J Virol 85, 9527-9542 (2011) (incorporated by reference herein) was used to determine the role of MPXV197 in viral dissemination, pathogenesis and induction of T cell responses. To rule out that MPXVΔ197 contained additional mutations compared to parental strain MPXV-US2003 we sequenced the genomes for both viruses by next generation (NextGen) sequencing. Within a margin of error (<3%) both WT and MPXVΔ197 matched the predicted sequence exactly (FIGS. 16A and 16B, Table 1).

TABLE 2
Location of SNPs in MPXV analyzed by NextGen sequencing.
Any SNP detected in >1% of reads at that position, with
at least 500 reads is shown. For each SNP, the frequency,
depth of coverage and predicted amino acid changes are shown.
All NT positions are reported relative to the wild-type sequence.
MPXV-US2003 did not contain SNPs >1%. All MPXVΔ197
SNPs >1% are located near or within the terminal repeats
(NT 1-8836 and 189945-198780) in the intergenic regions.

| Sample | NT | Ref | Read | | | |
|---|---|---|---|---|---|---|
| Name | Position | NT | NT | Reads | Coverage | Percent |
| MPXV Δ197 | 690 | C | T | 14 | 500 | 2.8 |
| MPXV Δ197 | 198091 | G | A | 9 | 509 | 1.77 |
| MPXV Δ197 | 193881 | A | C | 8 | 520 | 1.54 |
| MPXV Δ197 | 193881 | A | T | 8 | 520 | 1.54 |
| MPXV Δ197 | 193889 | G | T | 7 | 507 | 1.38 |
| MPXV Δ197 | 189689 | T | G | 7 | 509 | 1.38 |
| MPXV Δ197 | 193886 | T | C | 7 | 513 | 1.36 |
| MPXV Δ197 | 193882 | T | C | 7 | 520 | 1.35 |
| MPXV Δ197 | 189690 | G | A | 6 | 510 | 1.18 |
| MPXV Δ197 | 193884 | A | C | 6 | 517 | 1.16 |
| MPXV Δ197 | 4839 | A | C | 6 | 530 | 1.13 |

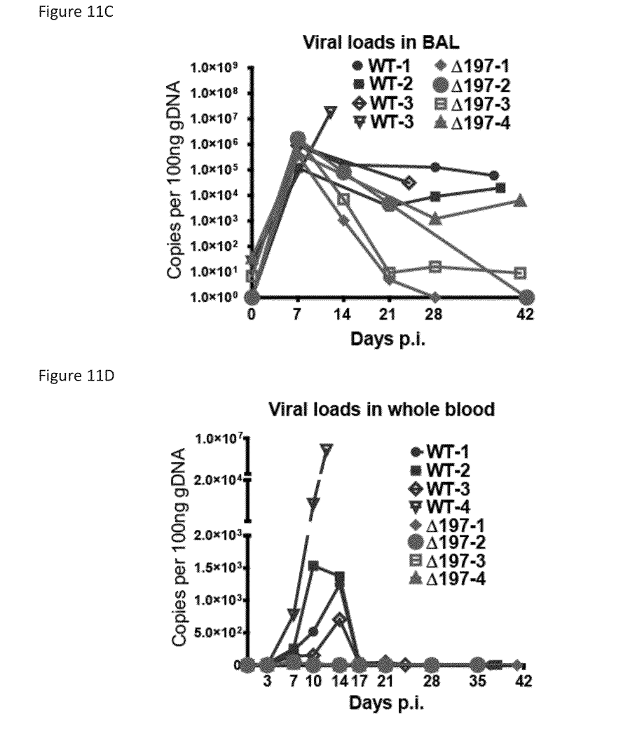

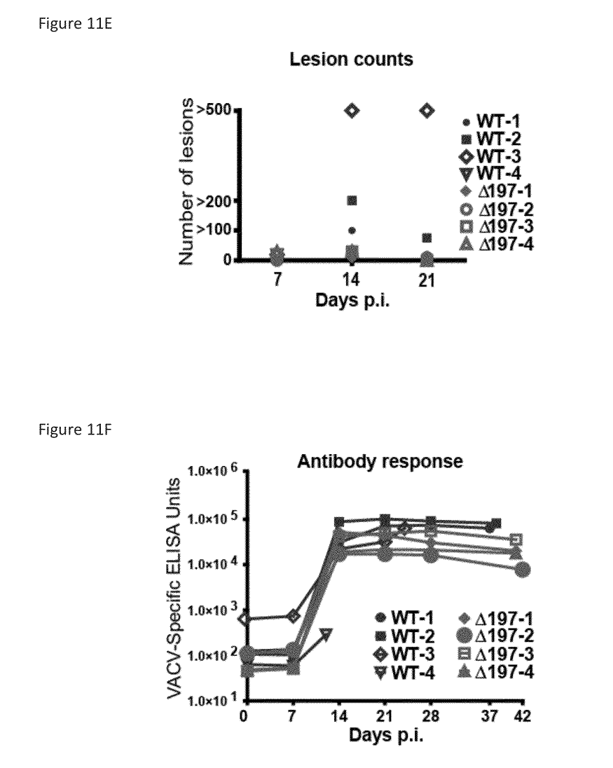

Since this analysis cannot distinguish between sequencing errors, misalignments (particularly in the repeat region) and actual mutations, it is likely that the actual percentage of correct genome sequences is substantially higher. A total of 8 RM were infected with MPXV-US2003 or MPXVΔ197 using intrabronchial inoculation of 2×10⁵ PFU (FIG. 11A), a dose at which MPXV-Zaire was non-lethal (Estep et al 2011 supra).

The clinicopathologic course of infection was followed by physical examination, biotelemetry to record body temperature and activity, O₂ tissue saturation, and development of cutaneous lesions. Blood and bronchoalveolar lavage (BAL) fluid samples were collected at defined days post infection (dpi) to determine the kinetics of virus replication and of the adaptive immune response. As shown in FIG. 6A-6E and Table 2, RM infected with MPXVΔ197 experienced a significantly shorter duration of fever (5 days compared to 20 days) (FIG. 6B), fewer skin lesions (FIG. 6E), and dramatically reduced morbidity and mortality.

TABLE 2
Skin lesion counts in RM infected with
MPXVUS2003 wild type and Δ197 mutant.

| Days | WT-1 | WT-2 | WT-3 | WT-4 | Δ197-1 | Δ197-2 | Δ197-3 | Δ197-4 |
|---|---|---|---|---|---|---|---|---|
| 7 | 0 | 0 | 20 | 20 | 0 | 0 | 20 | 30 |
| 14 | >100 | >200 | >500 | — | 10 | 20 | 30 | 30 |
| 21 | 20 | 75 | >> | — | 5 | 10 | 0 | 0 |

In fact, two of the MPXV-US2003-infected RM had to be euthanized due to deteriorating health whereas all four of the MPXVΔ197-infected RM spontaneously controlled the infection prior to termination of the experiment at days 41 and 42. Viral titers measured in the lungs were initially similar, reflecting the similar size of the inoculum, but lung titers of MPXVΔ197 fell significantly more rapidly compared to WT (FIG. 11C). An even more striking contrast was observed for viral titers in the blood where all RM infected with MPXV-US2003 showed significantly higher levels of viremia compared to MPXVΔ197 which was barely detectable (FIG. 11D). Interestingly, while uncontrolled viremia in both lungs and blood correlated with rapid deterioration of health in one animal (WT-4), the other animal that needed to be euthanized prematurely (WT-3) had a lower viremia in the blood but a higher number of lesions at days 14 and 21 compared to the remaining WT-infected RM (FIG. 11E). In contrast, low titers in the blood correlated with a generally mild disease and less than 30 lesions in MPXVΔ197-infected RM (FIG. 11E, Table 2). Decreased viral titers of MPXVΔ197 were also reflected in a decrease of antibody titers which tended to be lower than that of MPXV-US2003 although this was not statistically significant (FIG. 11F).

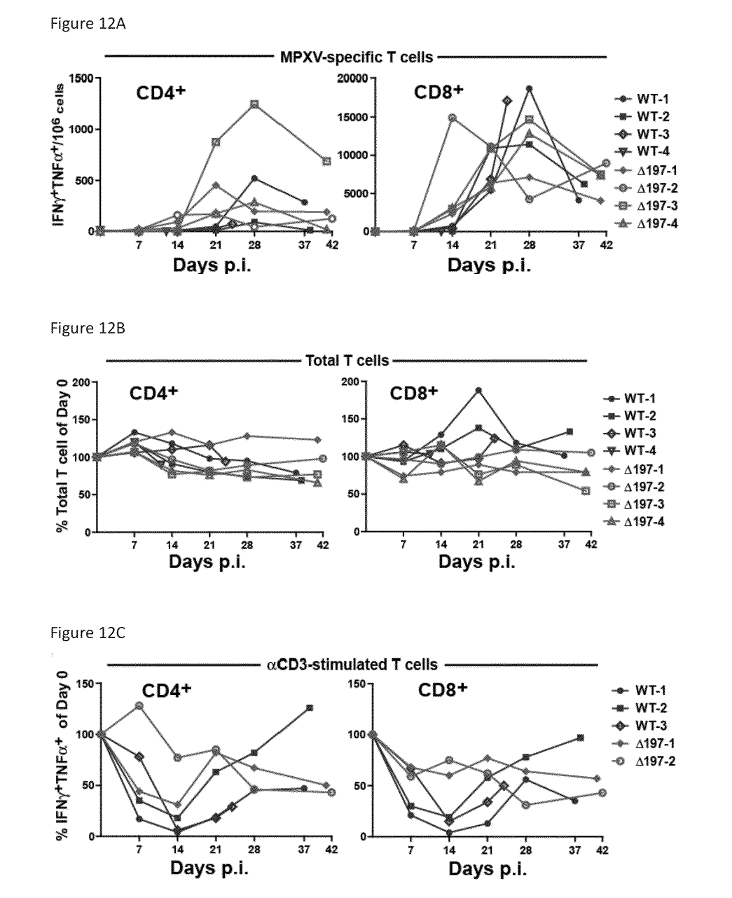

In stark contrast to the reduced virologic and disease parameters, poxvirus-specific T cell responses were detected earlier and were significantly higher at some of the earliest time points in RM infected with MPXVΔ197 compared to those infected with MPXV-US2003 (FIG. 12A). (Note that T cell responses were measured using VACV to avoid the T cell inhibitory effect of MPXV197). At day 14, all four MPXVΔ197-infected RM had a significantly higher frequency of poxvirus-specific CD8+ T cells in their blood compared to the 3 remaining WT-infected RM (FIG. 12A). Similarly, in 3 of 4 MPXVΔ197-infected RM the CD4+ T cell response was above background at days 7 and 14 whereas 0/4 or 2/3 WT-infected RM had detectable CD4+ and CD8+ T cells at these days. At day 21, the frequency of CD4+ T cells in all MPXVΔ197-infected RM was significantly higher than in WT-infected RM. The inverse correlation between viral titers and T cell responses in the blood is consistent with MPXV197 contributing to viral dissemination during the early phase of infection by delaying the onset of the cellular immune response.

To examine whether the T cell inactivation mediated by MPXV197 would result in a systemic suppression of T cell responses during viral infection in vivo, T cells in PBMC were stimulated with an anti-CD3 antibody. The data are limited to three WT and two MPXVΔ197-infected RM since two animals were missing samples and T cells from Δ197-3 were unresponsive to anti-CD3 stimulation. Although the overall frequency of T cells in the blood did not change during infection (FIG. 12B), there was a dramatic reduction in anti-CD3 responses of both CD4+ and CD8+ T cells from WT-infected RM at 7-21 dpi (FIG. 12C). This was particularly evident at day 14 which correlated with peak viremia in the blood of WT-1 and WT-2-infected animals (FIG. 12D). In contrast, this decrease was less pronounced for αCD3-stimulation of T cells in both MPXVΔ197-infected RM. Although not statistically significant due to the low sample size, these observations are consistent with MPXV197 contributing to a systemic suppression of T cell responses during peak viremia.

### Example 14

### Materials and Methods Used

Cells and Viruses:

Human foreskin fibroblasts (HFF), BEAS-2B human bronchial epithelium cells, human 143 cells, Chinese hamster ovary (CHO) cells, and human embryonic kidney (HEK) 293 cells were maintained in Dulbecco's modified Eagle's medium (DMEM, Mediatech, Manassas, Va.) supplemented with 10% fetal bovine serum (FBS, Hyclone Laboratories, Inc, Logan, Utah). Rhesus macaque (RM) B-lymphoblastoid cell line (BLCL) was grown in 10% FBS-RPMI 1640 medium (Hyclone Laboratories, Inc). Mtb specific T cell clones and monkey CM9-peptide specific T cell lines and were maintained as described in Loffredo J T et al, J Virol 81, 2624-2634 (2007); Lewinsohn D A et al, 2007 supra; and Heinzel A S et al, J Exp Med 196, 1473-1481; all of which are incorporated by reference herein. BSC40, African Green Monkey kidney cells were grown in minimum essential medium (MEM, Mediatech). Jurkat T cells clone JJK were grown in 10% FBS-RPMI 1640 medium (Hyclone Laboratories, Inc).

Vaccinia virus (VACV) Western Reserve strain, monkeypox virus (MPXV) strains Zaire and US2003, Cowpox virus (CPXV) Brighton Red strain were propagated in BSC40 cells maintained in 5% FBS MEM. The virus preparations were purified using a standard protocol Hruby D E et al, J Virol 29, 705-715 (1979) with minor modifications. Briefly, the infected cells were harvested, resuspended in 10 mM Tris-HCl (pH8.0), and lysed by three cycles of freezing-thawing followed by two cycles of sonication. Precleared cell lysate was layered onto 36% sucrose cushion and centrifuged at 40,000×g for 80 min. Pelleted virus particles were resuspended in 1 mM Tris-HCl (pH8.0) and titered. For complete genome sequencing and in-vivo studies, the virus was additionally purified by centrifugation (22,500×g, 40 min) through a 25% to 40% continuous sucrose gradient.

Human Subjects:

VACV-immune subjects provided informed written consent before signing research authorization forms that complied with the US Health Insurance Portability and Accountability Act (HIPAA) in addition to a medical history questionnaire. These studies were approved by the Institutional Review Board of OHSU.

Animals:

All animal studies were carried out in strict accordance with the recommendations in the Guide for the Care and Use of Laboratory Animals (8th edition, The National Academies Press) and the Animal Welfare Act (the National Institutes of Health Office of Laboratory Animal Welfare assurance number A3304-01). All animal procedures were performed according to protocols #0865 and #0731 approved by the Institutional Animal Care and Use Committee of the Oregon Health and Science University. Appropriate sedatives, anesthetics and analgesics were used during handling, and clinical and surgical procedures to ensure minimal pain, suffering and distress to animals. Female BALB/c mice at 5 months of age were purchased from The Jackson Laboratory. Mice were immunized intraperitoneally (i.p.) with 2×10⁶ PFU/mouse of VACV WR. On day 8 post inoculation, spleens were collected and used for studies of the T cell responses to CPXV BR wild type and mutants.

Eight adult female RM animals were utilized for in-vivo studies of the T cell responses to MPXV US2003 wild type (WT) and MPXVΔ197 mutant. Cohort 1 (WT) included animals 29437 (WT-1; 7 year-old), 29785 (WT-2; 10-year-old), 21111 (WT-3; 13 year-old), and 28689 (WT-4; 13-year-old). Cohort 2 (Δ197) included animals 29792 (Δ197-1; 8-year-old), 29398 (Δ197-2; 11-year-old), 29424 (Δ197-3; 13-year-old), 28664 (Δ197-4; 10-year-old). The animals were infected intrabronchially with 5×10⁵ PFU/animal of WT and the mutant viruses delivered in 1 ml of phosphate-buffered saline (PBS). Blood and bronchoalveolar lavage (BAL) samples were collected on the day of infection (day 0) and later on the days post infection indicated in FIG. 12A. Peripheral blood mononuclear cells (PBMC) were isolated from blood by centrifugation over Lymphocyte Separation Media. Body temperature and physical activity were monitored via telemetry implants (Mini Mitter, Bend, Oreg.).

Construction of Expression Plasmids:

Codon-optimized sequences of the C-terminal 3×FLAG (DYKDHDGDYKDHDIDYKDDDDK) fusions of MPXV197 and VARV B22 proteins were synthesized at GenScript (Piscataway, N.J.). MPXV197 N-terminal Flag fusion was constructed by removing 3×FLAG sequence from the C-terminus of the protein and inserting it downstream of the predicted signal sequence after the amino acid E₂₁. All coding sequences were cloned into pCDNA3.1 vector (Life Technologies). Additionally, MPXV 197-CFlag and VACV B22-CFlag coding sequences were sub-cloned in pAdtet7 shuttle vector (Altschuler Y et al, J Cell Biol 143, 1871-1881 (1998); incorporated by reference herein) under a tetracycline (tet) regulated promoter. The resulting plasmids were used for construction of the recombinant adenoviral vectors. To achieve the protein expression these viruses were co-infected with Ad-tTA virus expressing tet-transactivator (tTA) protein (Streblow D N et al, Cell 99, 511-520 (1999) incorporated by reference herein). In vitro synthesis of VARV B22R ORF and all in vitro experiments using B22R-expressing constructs were approved by the World Health Organization (WHO).

Recombinant Viruses:

Recombinant MPXV:

All work with this virus and the recombinant derivative was conducted in accordance with institutional guidelines for biosafety at OHSU. MPXV deletion mutants in US2003 strain were generated via homologous in vivo recombination (Boyle D B and Coupar B E Gene 65, 123-128 (1988); incorporated by reference herein) replacing up to ˜10 kb fragments with a GFP-GPT cassette. Recombination plasmids were constructed by splicing regions upstream and downstream of the indicated ORFs to the 5′ and 3′ termini of the cassette expressing green-fluorescent protein (GFP) and guanine-hypoxanthine phosphoribosyltransferase (GPT) using spice overlap extension by PCR technique (Horton R M et al, Gene 77, 61-68 (1989); incorporated by reference herein). The nucleotide sequences were PCR-amplified from MPXV US2003 genomic DNA and pT7 E/L EGFP-GPT vector (Cameron C M et al, Virology 337, 55-67 (2005) and subsequently fused by PCR using primers described in the attached sequence listing. The resultant fragments were cloned into pCR2.1-TOPO-TA vector (Life Technologies, Grand Island, N.Y.) using the manufacturer's protocol.

For in vivo recombination, BSC40 cells transfected with a recombination plasmid were infected with MPXV US2003 at a multiplicity of infection (MOI) of 0.1 and incubated for 48 h. Recovered virus was passaged twice in GPT selection medium, 5% FBS MEM-32 μg mycophenolic acid/ml-250 μg xanthine/ml-15 μg hypoxanthine/ml and then plaque purified. The resultant recombinant viruses were amplified and purified by centrifugation through sucrose. To verify the deletion and the absence of contaminating wild-type virus, viral genomic DNA was purified with DNeasy kit (QIAGEN, Valencia, Calif.) and tested by PCR using primers specific to the flanking regions. Additionally, to confirm that no other major deletions or mutations were acquired during construction of Δ197 mutant, genomic DNA of both the wild type and the mutant viruses was sequenced by using complete genome sequencing. The Recombinant MPXVΔORF184 deletion mutant in Zaire strain was generated by replacing the CPXV203 orthologue ORF 184 with a GFP-GPT cassette using the same protocol.

Recombinant CPXV: CPXV Δ12Δ203-221 is a spontaneously derived mutant from previously described CPXV Δ12Δ203 recombinant virus (Alzhanova D et al, 2009 supra). Initially ORFs 12 and 203 were replaced with (E/L Pr.)GFP-(7.5K Pr.)GPT and (7.5K Pr.)Neo-(p4b Pr.)RFP expressing cassettes, respectively. Upon passaging, ORFs 204-221 were replaced with an inverted copy of ORFs 10-11, likely due to homologous recombination between the inverted copies of vaccinia 7.5K promoter driving expression of both GPT and Neo and the inverted terminal repeats of the viral genome. The duplication of terminal ORFs was confirmed by PCR and sequencing of genomic DNA.

CPXVΔ12 A203, a mutant virus with deleted ORFs 12 and 203 and CPXV Δ203 in which ORF 203 was replaced with a GFP-expressing cassette were described in Byun et al, 2009 supra and Byun et al 2007 supra.

CPXV Δ204-221 (A694) is a spontaneously generated white pock variant (W3 variant) of CPXV (Brighton red strain) isolated and initially described in Pickup D J et al, Proc Natl Acad Sci USA 81, 6817-6821 (1984); incorporated by reference herein. Subsequent sequence analysis showed that in comparison to the genome of the wild-type virus, the genome of this variant has lost the 33.7 kb region from nucleotide 190,832 to the right-hand end of the genome (nucleotide 224,499), with the deleted region replaced by an inverted copy of the left-hand end of the genome encompassing nucleotides 1-15, 461.

CPXVΔ219 (A618) mutant that lacks 96% of the 5759-nucleotide coding region of CPXV219 was constructed via homologous recombination in-vivo as described above. Plasmid p1889 was generated containing GPT gene under the control of the vaccinia virus p7.5 promoter flanked by XmaI sites within a pGem7zf vector as described in Panus J F et al, Proc Natl Acad Sci USA 99, 8348-8353 (2002); incorporated by reference herein). The gpt gene was then flanked by the XhoI-MfeI fragment (residues 205202-205719) and the HinPI-ClaI fragment (residues 209953-211788) at the 5′ and 3′ ends of the CPXV219 gene, to create plasmid p1903, which was used to create the mutant virus.

Recombinant VACV: VACV-219 corresponds to recombinant VACV A625 that expresses the CPXV219 gene under the control of the bacteriophage T7 RNA polymerase. The CPXV219 coding region was placed into the insertion vector pTM1 (Moss B et al, Nature 348, 91-92 (1990); incorporated by reference herein) by first inserting PCR products of the 5′ and 3′ ends of the CPXV219 coding region such that the initiation codon was at the NcoI site in pTM1, an XhoI site was downstream of the stop codon, and unique restriction sites SphI and BssHI present at the two ends of the coding region were present in the modified pTM1 plasmid. The PCR modifications were done using primers NcoI-219-5′-SphI-F and NcoI-219-5′-SphI-R to produce the 5′ end fragment containing the SphI site, with primers BssH1-219-3′-XhoI-F and BssH1-219-3′-XhoI-R (Table S1) to produce the 3′ end fragment containing the unique BssHI site. Then the 5526 kbp SphI-BssH1 fragment of cloned CPXV DNA in plasmid p1906 containing the entire CPXV219 gene was inserted into the modified pTM1 vector to create plasmid p1951 in which the full-length CPXV219 gene is under the control of the T7 promoter. This plasmid was used to create a recombinant VACV-219 via homologous recombination in-vivo as described in Mackett M et al, J Virol 49, 857-864 (1984), incorporated by reference herein. The expression of CPXV219 in cells co-infected with VACV-219 and VTF7-3 (Feurst T R et al, Mol Cell Biol 7, 2538-2544 (1987); incorporated by reference herein), a VACV expressing the phage T7 polymerase, was confirmed by immunoprecipitation of proteins metabolically labeled with [³⁵S] methionine and immunoblot (FIG. 11F). Since T cell inhibition by VACV-219 was observed regardless of co-infection by VTF7-3, we used single infection in our T cell assays. T7-polymerase-independent expression of T7-promoter-driven poxviral genes has been reported in Vennema H et al, Gene 108, 201-209 (1991); incorporated by reference herein.

Rabbit polyclonal antisera used in these assays were raised against CPXV219 protein expressed as a glutathione S-transferase (GST) fusion protein in E. coli from a pGEX-3× vector as described in Smith D B and Corcoran L M, Curr Protoc Mol Biol Ch. 17, Unit 16-17 (incorporated by reference herein). For this construct, primers were used to insert into the pGEM-3× plasmid a BamHI-XmaI linker containing 5′ end of the CPXV219 gene in-frame with the GST coding region, and including XhoI and SphI sites into which the remainder of the coding region of CPXV219 was inserted from an XhoI-SphI DNA fragment obtained from p1951.

Virus Titering:

BSC40 cells were plated into 6-well plates at 30% confluency. The next day, the cells were infected with 250 μl of a serial 10-fold dilution of the virus preparation or the infected cell lysate. At 1 hour post infection, the cells were overlaid with 0.5% agarose (Life Technologies, Grand Island, N.Y.)-EMEM (Quality Biological, Gaithersburg, Md.) and incubated for 5 days at 37° C. The cells were fixed with 75% methanol-25% Acetic Acid for 20 min and stained with 0.1% crystal violet-30% ethanol.

Next Generation Sequencing of MPXV Genomes:

Genomic DNA of the wild type MPXV and Δ197 mutant was isolated using DNeasy kit from the virus preparations purified through a 25% to 40% continuous sucrose gradient. DNA libraries were generated by the OHSU Massively Parallel Sequencing Shared Resource (MPSSR) core using the TruSeq DNA Sample Preparation kit (Illumina, San Diego, Calif.). The sequencing was performed using a MiSeq sequencer (Illumina) at the Molecular and Cellular Biology (MCB) core at the ONPRC. The resulting DNA reads were aligned to the published genome sequence of MPXV-USA2003-039 (GenBank accession # DQ11157). Illumina sequence data were processed using a custom analysis pipeline written by B.N.B. This pipeline has been made available as a module for LabKey Server, an open-source platform for the management of scientific data (Nelson E K et al, BMC Bioinformatics 12, 71 (2011); incorporated by reference herein). The SequenceAnalysis module provides a web-based interface to initiate analyses, manage data, and view results. The source code behind this pipeline is available in a subversion repository. Raw reads were trimmed by sequence quality using Trimmomatic (Lohse M et al, Nucleic Acids Res 40, 622-627 (2012); incorporated by reference herein) and aligned against the reference genome using BWA-SW (Li H and Durbin R, Bioinformatics 25, 2078-2079 (2009); incorporated by reference herein). Single Nucleotide Polymorphisms (SNPs) between reads and the reference sequences were scored with scripts that utilized SAMtools, picard tools (http://picard.sourceforge.net), and bioperl (Li H et al, Bioinformatics 25, 2078-2079 (2009) and Stajich J E et al, Genome Res 12, 1611-1618 (2002); incorporated by reference herein).

Pulse-Chase Labeling and Immunoprecipitation:

CHO cells were transduced with either Ad-tTA (25 MOI) or Ad-197 (20 MOI) and Ad-tTA (5 MOI). At 24 h post transduction (p.t.), the cells were washed with PBS, overlaid with DMEM (Cys⁻/Met⁻), and incubated for 1.5 hours. The cells were pulsed with 300 μCi/10⁶ cells for 45 min and the label was chased for the indicated time intervals. CHO cells were washed with ice-cold PBS and lysed with ice-cold PBS-1% NP-40 buffer. Cell lysates were pre-cleared with agarose beads and immunoprecipitated with αFLAG Ab conjugated to agarose beads (Sigma-Aldrich, St. Louis, Mo.). The samples were eluted from the beads with 50 mM NaOAc-0.15% SDS buffer (10 min, 98° C.) and treated with EndoH (Roche Diagnostics, Indianapolis, Ind.) or PNGase (New England Biolabs, Ipswich, Mass.) according to the manufacturer's protocols. The samples were separated on a 6% polyacrylamide gel.

Immunoblot:

CHO cell lysates or immunoprecipitated samples were separated on 6% polyacrylamide gels and transferred onto Immobilon PVDF membranes (EMD Millipore, Billerica, Mass.). The membrane was blocked with 5% skim milk in PBS-0.05% Tween 20 (PBST) buffer and blotted with αFLAG Ab (Sigma-Aldrich, 1:500) and secondary HRP-conjugated mouse TrueBlot Ab (eBioScience, San Diego, Calif.) diluted in 5% skim milk-PBST. The immunoblots were developed with SuperSignal West Pico Chemiluminescent Substrate kit (Thermo Fisher Scientific, Rockford, Ill.).

Cell-Surface Biotinylation:

CHO cells grown in T75 flasks to 80% confluency were transduced with either Ad-tTA alone (25 MOI) or Ad-197 (20 MOI) and Ad-tTA (5 MOI) or Ad-B22R (20 MOI) and AdtTA (5 MOI). After 24 h incubation, the cells were washed twice with PBS and biotinylated using Pierce Cell Surface Protein Isolation kit (Thermo Fisher Scientific, Rockford, Ill.) according to the manufacturer's protocol. Biotinylated proteins were immunoprecipitated with NeutrAvidin agarose resin provided with the kit, separated on 6% PAGE gel, and blotted with αFLAG Ab.

Immunofluorescence and Confocal Laser Scanning Microscopy:

CHO cells were plated on glass coverslips in 12-well plates at 50% confluency. The next day the cells were transfected with 500 ng of indicated plasmids using Lipofectamine 2000 (Life Technologies) according to the manufacturer's protocol. At 24 h post transfection, the cells were washed with ice-cold PBS, fixed with 4% paraformaldehyde, and permeabilized with 0.2% Triton X100. The samples were blocked with 2% bovine serum albumin (BSA)-PBS (P-BSA, pH 7.4) and stained with primary mouse αFLAG Ab (1:1000) and secondary anti-mouse to Alexa Fluor 594 (1:1000, Life Technologies) diluted in 2% P-BSA. The coverslips were mounted on slides in ProLong Gold antifade reagent with 4,6-diamidino-2-phenylindole (DAPI; Life Technologies) and analyzed with Leica TCS SP laser scanning microscope.

T Cell assays:

Human and Rhesus Macaque PBMC:

T cell responses in PBMC were measured as described below. Briefly, PBMC were infected with or without the indicated viruses (MOI of 0.3-0.6). After 12 hours of incubation, Brefeldin A (BFA; ICN Biomedicals Inc., Costa Mesa, Calif.) was added at a final concentration of 2 μg/mL for an additional 6 hours. For αCD3-stimulation, PBMC were infected with or without the indicated viruses (MOI 0.3-0.6) for 12 h prior to incubation with plate-bound αCD3 (0.15 μg/ml, 100 μl/well, clone HIT3a, NA/LE; BD Biosciences PharMingen PharMingen, San Diego, Calif.) for 6 h in the presence of BFA. RM PBMC were incubated with soluble αCD3 (0.1 μg/well, clone FN 18) for 6 h in the presence of BFA. The cells were stained overnight at 4²C with Ab specific for CD8β (clone 2ST8.5H7, Beckman Coulter, Brea Calif.) and CD4 (clone L200, BD Biosciences PharMingen, San Diego, Calif.). Cells were fixed with 2% formaldehyde in PBS, permeabilized with PermWash (0.1% saponin and 1% FBS in PBS) and stained intracellularly using Ab to IFNγ (clone 4S.B3, eBioscience Inc., San Diego, Calif.) and TNFα(clone Mab11, eBioscience). Samples were acquired on an LSR Fortessa (BD Biosciences) using FACS-DIVA software (BD Biosciences) and analyzed using FlowJo software (Tree Star). Non-viable cells were excluded using a live cell gate based on the viability stain (LIVE/DEAD Fixable Dead Cell Stain, Life Technologies), followed by an optimized lymphocyte gate based on forward and side scatter characteristics. The number of virus-specific IFNγ⁺/TNFα⁺ T cells was determined after gating on live CD4⁻CD8β⁺ or CD4⁺ CD8β⁻ T cells and subtracting the number of IFNγ⁺TNFα⁺ events from uninfected or unstimulated cultures.

Human Mtb-Specific T Cell Clones:

To study T cell responses in the presence of MPXV 197, BEAS-2b cells were infected with Ad-tTA (8 MOI) or Ad-197 (6 MOI) and Ad-tTA (1.7 MOI) for 72 h. Alternatively, to study T cells responses in the presence of CPXV 219 the cells were pretreated with 10 μM ST246 provided by SIGA Technologies (Corvallis, Oreg.) and infected with VACV wild type or VACV-219 recombinant virus (2 MOI, 10 μM ST246) for 2 h. The ST246 drug was included at all other stages of the experiments utilizing VACV infected BEAS-2b cells. Indicated Mtb-specific T cell clones were co-incubated with infected BEAS-2b cells for 3 h or overnight and then stimulated with peptide CFP10₂₋₁₂ (clone D466 D6), pronase digested Mtb cell wall (clone D160 1-23), or phytohemagglutinin (PHA) in αIFN-γ Ab (clone 1-D1K, MABTECH AB, Nacka Strand, Sweden) coated ELISPOT plates. The staining was detected with αIFN-γ Ab conjugated to horseradish peroxidase (HRP) clone7-B6-1, MABTECH AB and developed using ABC Vectastain-Elite kit (Vector Laboratories, Burlingame, Calif.).

Rhesus CM9 Peptide-Specific T Cell Lines:

To study T cell responses in the presence of ST246, HFF cells were pretreated with 10 μM ST246 and infected with indicated viruses (2 MOI, 10 μM ST246) for 2 hours. ST246 was included in all subsequent steps of the T cell assay at the same concentration. For the experiments utilizing Ad-MPXV 197, CHO cells were infected with either Ad-tTA (25 MOI) or Ad-197 (20 MOI) and Ad-tTA (5 MOI) for 24 h. The infected cells were overlaid with T cells for specific time periods or overnight. After co-incubation, T cells were collected, washed, and transferred into a fresh plate for stimulation with either autologous BLCL cells pulsed with CM9 peptide (SIVgag₁₈₁₋₁₈₉, CTPYDINQM; Genscript, Piscataway, N.J.) or phorbol 12-myristate 13-acetate (PMA)/lonomycin in the presence of BFA for 5 hours. The cells were washed with PBS and stained with αCD8 (clone SK1, BD Biosciences) and αCD4 (clone L200, BD Biosciences) Ab and LIVE/DEAD Fixable Dead Cell Stain (Life Technologies) for 30 min at room temperature. The cells were fixed and permeabilized with BD Cytofix/Cytoperm (BD Biosciences) and stained intracellularly with Ab specific to TNFα (clone 6.7, BD Biosciences), IFNγ (clone 25723.11, BD Biosciences), and CD3 (clone SP34-2, BD Biosciences). The samples were analyzed by flow cytometry as described above.

Murine T Cell Assay:

Poxvirus-specific T cell responses in murine splenocytes were measured. Briefly, splenocytes isolated from VACV-infected mice (2×10⁶ PFU/mouse) at 8 days post infection were stimulated with A20 cells infected with CPXV or VACV (MOI=5, 16 h) in the presence of Brefeldin A for 6 h. Cells were stained overnight at 4° C. with αCD3E and αCD4 Ab's (clones 145-2C11 and RM 4-5, respectively, BD Biosciences), αCD8 Ab (clone 5H10, Life Technologies), Fc Block (BD Biosciences) and mouse IgG (Sigma). The next day, the cells were washed, fixed, and permeabilized with BD Cytofix/Cytoperm (BD Biosciences) followed by intracellular staining with Ab to IFNγ (clone XMG1.2, BD Biosciences PharMingen) and TNFα (clone MP6-XT22, BioLegend, San Diego, Calif.). The samples were analyzed by flow cytometry as described above. Non-viable cells were excluded using a live cell gate based on Aqua staining, gated for lymphocytes based on forward and side scatter characteristics followed by gating for CD3ε⁺. Next, CD3ε⁺ T cells were gated on either CD4⁺ or CD8⁺ and IFNγ⁺TNFα⁺ T cells were quantified. Background IFNγ⁺TNFα⁺ events from uninfected samples were subtracted. T cell responses to CPXV and CPXV deletion mutants were normalized to VACV.

Tetramer Binding:

MaMu-A*01 CM9 tetramer was conjugated to Allophycocyanin (APC) using ProZyme PhycoPro GT5 APC kit (Prozyme, Hayward, Calif.) according to the manufacturer's protocol. Monkey CM9-peptide specific T cells recovered after co-incubation with Ad-197/Ad-tTA or Ad-tTA only infected CHO cells (described above) were incubated with the tetramer for 1 h at 37° C. and stained with LIVE/DEAD Fixable Dead Cell Stain (Life Technologies) and Ab specific to CD95 (clone DX2, BD Biosciences), CD28 PE (clone L293, BD Biosciences), CD45 (clone D058-1283, BD Biosciences), CD8 (clone SK1, BD Biosciences), and CD3 (clone SP34-2, BD Biosciences) for 30 minutes at room temperature. The cells were fixed with 2% paraformaldehyde and analyzed by flow cytometry as described above.

ELISA:

Orthopox-specific enzyme-linked immunosorbent assay (ELISA) was performed using whole-VACV lysate (inactivated by pre-treatment with 3% H₂0₂ for 2 hours. An internal positive control was included on each plate to normalize between plates and between assays performed on different days. Antibody titers were determined by log-log transformation of the linear portion of the curve, using 0.1 optical density units as the endpoint and performing conversion on final values.

1. A recombinant expression vector comprising:
a nucleic acid sequence that encodes a polypeptide of SEQ ID NO: 1 or a homolog thereof and a heterologous promoter operably linked to the nucleic acid sequence.

2. The expression vector of claim 1 wherein the nucleic acid sequence encodes a polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

3. The expression vector of claim 1 wherein the nucleic acid sequence encodes a polypeptide that is 95% identical to SEQ ID NO: 1, SEQ ID NO: 2, SEQ ID NO: 3, SEQ ID NO: 4, or SEQ ID NO: 6.

4. The expression vector of claim 1 wherein the nucleic acid sequence is a codon optimized sequence for expression in mammalian cells.

5. The expression vector of claim 4 wherein the nucleic acid sequence is SEQ ID NO: 8 or SEQ ID NO: 9.

6. The expression vector of claim 1 wherein the expression vector is a plasmid vector or heterologous viral vector.

7. The expression vector of claim 6 wherein the heterologous viral vector is an adenoviral vector.

8. The expression vector of claim 1 wherein the promoter is an inducible or constitutive promoter active in a mammalian cell.

9. A method of inhibiting a CD4+ or CD8+ T cell, the method comprising:
administering a pharmaceutical composition comprising the recombinant expression vector of claim 1 to cells of a subject thereby causing the cells to express SEQ ID NO: 1 or a homolog thereof.

10. The method of claim 9 wherein administering the pharmaceutical composition to the cells of the subject occurs in vivo.

11. The method of claim 10 wherein the pharmaceutical composition is administered locally

12. The method of claim 10 wherein the pharmaceutical composition is administered systemically.

13. The method of claim 10 wherein the pharmaceutical composition is administered via injection.

14. The method of claim 9 wherein administering the pharmaceutical composition to the cells of the subject occurs ex vivo, the method further comprising administering cells expressing SEQ ID NO: 1 or the homolog thereof back to the subject.
